SEQUENCE LISTING (82 sequences)

SEQ ID NO: 1 - RNA, 27 nt, Artificial Sequence
  Synthetic Sequence - siCtBP1 (mouse)
ucuuccacagugugacugcguuauuuu

SEQ ID NO: 2 - RNA, 24 nt, Artificial Sequence
  Synthetic Sequence - siCtBP2 (mouse)
gccuuuggauucagcgucauauuu

SEQ ID NO: 3 - RNA, 21 nt, Artificial Sequence
  Synthetic Sequence - siCtBP1 (human)
acgacuucaccgucaagcauu

SEQ ID NO: 4 - RNA, 23 nt, Artificial Sequence
  Synthetic Sequence - siCtBP2 (human)
  misc_feature (21)..(21): n is a, c, g, or u
  misc_feature (23)..(23): n is a, c, g, or u
gcgccuuggucaguaauagdndn

SEQ ID NO: 5 - protein, 49 aa, Artificial Sequence
  Synthetic Sequence - Pep1-E1AWT
GSHMKETWWETWWTEWSQPKKKRKVLEEPGQPLDLSCKRPRDYKDDDDK

SEQ ID NO: 6 - protein, 49 aa, Artificial Sequence
  Synthetic Sequence - Pep1-E1AMut
GSHMKETWWETWWTEWSQPKKKRKVLEEPGQPLDELCKRPRDYKDDDDK

SEQ ID NO: 7 - protein, 29 aa, Artificial Sequence
  Synthetic Sequence - Tat-E1A
GRKKRRQRRRPPQLEEPGQPLDLSCKRPR

SEQ ID NO: 8 - protein, 16 aa, Artificial Sequence
  Synthetic Sequence - pAntp
RQIKIWFQNRRMKWKK

SEQ ID NO: 9 - protein, 9 aa, Artificial Sequence
  Synthetic Sequence - Arg9
RRRRRRRRR

SEQ ID NO: 10 - protein, 16 aa, Artificial Sequence
  Synthetic Sequence - pLsL
RVIRVWFQNKRCKDKK

SEQ ID NO: 11 - protein, 5 aa, Artificial Sequence
  Synthetic Sequence - E1A-5
  misc_feature (2)..(2): Xaa can be any naturally occurring amino acid
PXDLS

SEQ ID NO: 12 - protein, 7 aa, Artificial Sequence
  Synthetic Sequence - E1A-7
  misc_feature (2)..(2): Xaa can be any naturally occurring amino acid
  misc_feature (6)..(6): Xaa can be any naturally occurring amino acid
PXDLSXK

SEQ ID NO: 13 - protein, 14 aa, Artificial Sequence
  Synthetic Sequence - E1A-14eg
EQTVPVDLSVARPR

SEQ ID NO: 14 - protein, 14 aa, Artificial Sequence
  Synthetic Sequence - E1A-14gg
GGDGPLDLCCRKRP

SEQ ID NO: 15 - protein, 14 aa, Artificial Sequence
  Synthetic Sequence - E1A-14pt
PTDEPLNLSLKRPR

SEQ ID NO: 16 - protein, 14 aa, Artificial Sequence
  Synthetic Sequence - E1A-14ep
EPGQPLDLSCKRPR

SEQ ID NO: 17 - protein, 22 aa, Artificial Sequence
  Synthetic Sequence - dNP2
KIKKVKKKGRKKIKKVKKKGRK

SEQ ID NO: 18 - protein, 23 aa, Artificial Sequence
  Synthetic Sequence MH-1
RRWRRWNRFNRRRGGPIDLSKKA

SEQ ID NO: 19 - protein, 18 aa, Artificial Sequence
  Synthetic Sequence - MH-2
RRRRRRRRGGPIDLSKKA

SEQ ID NO: 20 - protein, 13 aa, Artificial Sequence
  Synthetic Sequence - MH-3
RRRRRRRRPIDLS

SEQ ID NO: 21 - protein, 14 aa, Artificial Sequence
  Synthetic Sequence - MH-4
RRRRRGGPIDLSKK

SEQ ID NO: 22 - protein, 16 aa, Artificial Sequence
  Synthetic Sequence - MH-6
RRRRRRRRPIDLSKKA

SEQ ID NO: 23 - protein, 17 aa, Artificial Sequence
  Synthetic Sequence - MH-7
RRRRRRRRGGPIDLSKK

SEQ ID NO: 24 - protein, 11 aa, Artificial Sequence
  Synthetic Sequence - Tat47-57
YGRKKRRQRRR

SEQ ID NO: 25 - protein, 14 aa, Artificial Sequence
  Synthetic Sequence - Tat47-60
YGRKKRRQRRRPPQ

SEQ ID NO: 26 - protein, 13 aa, Artificial Sequence
  Synthetic Sequence - Tat48-60
GRKKRRQRRRPPQ

SEQ ID NO: 27 - protein, 14 aa, Artificial Sequence
  Synthetic Sequence -  Tat48-61
GRKKRRQRRRPPQQ

SEQ ID NO: 28 - protein, 9 aa, Artificial Sequence
  Synthetic Sequence - Tat49-57
RKKRRQRRR

SEQ ID NO: 29 - protein, 18 aa, Artificial Sequence
  Synthetic Sequence -  - SynB1
RGGRLSYSRRRFSTSTGR

SEQ ID NO: 30 - protein, 10 aa, Artificial Sequence
  Synthetic Sequence -  - SynB3
RRLSYSRRRF

SEQ ID NO: 31 - protein, 17 aa, Artificial Sequence
  Synthetic Sequence - SynB4
AWSFRVSYRGISYRRSR

SEQ ID NO: 32 - protein, 17 aa, Artificial Sequence
  Synthetic Sequence - SynB5
TGGRLAYLRRRWAVLGR

SEQ ID NO: 33 - protein, 19 aa, Artificial Sequence
  Synthetic Sequence - Angiopep-2
PFFYGGSGGNRNNYLREEY

SEQ ID NO: 34 - protein, 19 aa, Artificial Sequence
  Synthetic Sequence - Angiopep-5
RFFYGGSRGKRNNFRTEEY

SEQ ID NO: 35 - protein, 12 aa, Artificial Sequence
  Synthetic Sequence - FGF4
AAVLLPVLLAAP

SEQ ID NO: 36 - protein, 39 aa, Artificial Sequence
  Synthetic Sequence - RDP
KSVRTWNEIIPSKGCLRVGGRCHPHVNGGGRRRRRRRRR

SEQ ID NO: 37 - protein, 20 aa, Artificial Sequence
  Synthetic Sequence - TAT-HA
YGRKKRRQRRRYPYDVPDVA

SEQ ID NO: 38 - protein, 21 aa, Artificial Sequence
  Synthetic Sequence - ARF(1-22)
VRRFLVTLRIRRACGPPRVRV

SEQ ID NO: 39 - protein, 28 aa, Artificial Sequence
  Synthetic Sequence - BPrPr(1-28)
MVKSKIGSWILVLFVAMWSDVGLCKKRP

SEQ ID NO: 40 - protein, 28 aa, Artificial Sequence
  Synthetic Sequence - P28
LSTAADMQGVVTDGMASGLDKDYLKPDD

SEQ ID NO: 41 - protein, 24 aa, Artificial Sequence
  Synthetic Sequence - Bac7 (Bac1-24)
RRIRPRPPRLPRPRPRPLPFPRPG

SEQ ID NO: 42 - protein, 17 aa, Artificial Sequence
  Synthetic Sequence - C105Y
CSIPPEVKFNKPFVYLI

SEQ ID NO: 43 - protein, 6 aa, Artificial Sequence
  Synthetic Sequence - PFVYLI
PFVYLI

SEQ ID NO: 44 - protein, 21 aa, Artificial Sequence
  Synthetic Sequence - Buforin II
TRSSRAGLQFPVGRVHRLLRK

SEQ ID NO: 45 - protein, 16 aa, Artificial Sequence
  synthetic sequence - DVP3
RKKRRRESRKKRRRES

SEQ ID NO: 46 - protein, 17 aa, Artificial Sequence
  Synthetic Sequence - DPV6
GRPRESGKKRKRKRLKP

SEQ ID NO: 47 - protein, 15 aa, Artificial Sequence
  Synthetic Sequence - DPV7
GKRKKKGKLGKKRDP

SEQ ID NO: 48 - protein, 17 aa, Artificial Sequence
  Synthetic Sequence - DPV7b
GKRKKKGKLGKKRPRSR

SEQ ID NO: 49 - protein, 18 aa, Artificial Sequence
  Synthetic Sequence - DPV3/10
RKKRRRESRRARRSPRHL

SEQ ID NO: 50 - protein, 19 aa, Artificial Sequence
  Synthetic Sequence - DPV10/6
SRRARRSPRESGKKRKRKR

SEQ ID NO: 51 - protein, 19 aa, Artificial Sequence
  Synthetic Sequence - DPV1047
VKRGLKLRHVRPRVTRMDV

SEQ ID NO: 52 - protein, 18 aa, Artificial Sequence
  Synthetic Sequence - DPV1048
VKRGLKLRHVRPRVTRDV

SEQ ID NO: 53 - protein, 14 aa, Artificial Sequence
  Synthetic Sequence - DPV10
SRRARRSPRHLGSG

SEQ ID NO: 54 - protein, 16 aa, Artificial Sequence
  Synthetic Sequence - DPV15
LRRERQSRLRRERQSR

SEQ ID NO: 55 - protein, 22 aa, Artificial Sequence
  Synthetic Sequence - DPV15b
GAYDLRRRERQSRLRRRERQSR

SEQ ID NO: 56 - protein, 30 aa, Artificial Sequence
  Synthetic Sequence - GALA
WEAALAEALAEALAEHLAEALAEALEALAA

SEQ ID NO: 57 - protein, 21 aa, Artificial Sequence
  Synthetic Sequence - C-beta
KGSWYSMRKMSMKIRPFFPQQ

SEQ ID NO: 58 - protein, 20 aa, Artificial Sequence
  Synthetic Sequence - preC-gamma
KTRYYSMKKTTMKIIPFNRL

SEQ ID NO: 59 - protein, 20 aa, Artificial Sequence
  Synthetic Sequence - C-aplpha-E
RGADYSLRAVRMKIRPLVTQ

SEQ ID NO: 60 - protein, 24 aa, Artificial Sequence
  Synthetic Sequence - hCT (9-32)
LGTYTQDFNKFHTFPQTAIGVGAP

SEQ ID NO: 61 - protein, 12 aa, Artificial Sequence
  Synthetic Sequence - HN-1
TSPLNIHNGQKL

SEQ ID NO: 62 - protein, 12 aa, Artificial Sequence
  Synthetic Sequence - Influenza virus nucleoprotein (NLS)
NSAAFEDLRVLS

SEQ ID NO: 63 - protein, 30 aa, Artificial Sequence
  Synthetic Sequence - KALA
WEAKLAKALAKALAKHLAKALAKALKACEA

SEQ ID NO: 64 - protein, 16 aa, Artificial Sequence
  Synthetic Sequence - K-FGF
AAVALLPAVLLALLAP

SEQ ID NO: 65 - protein, 10 aa, Artificial Sequence
  Synthetic Sequence - Ku70
VPMLKPMLKE

SEQ ID NO: 66 - protein, 18 aa, Artificial Sequence
  Synthetic Sequence - MAP
KLALKLALKALKAALKLA

SEQ ID NO: 67 - protein, 27 aa, Artificial Sequence
  Synthetic Sequence - MPG P-beta
GALFLGFLGAAGSTMGAWSQPKKKRKV

SEQ ID NO: 68 - protein, 27 aa, Artificial Sequence
  Synthetic Sequence - MPG P-alpha
GALFLAFLAAALSLMGLWSQPKKKRRV

SEQ ID NO: 69 - protein, 16 aa, Artificial Sequence
  Synthetic Sequence - MPM (IP/K-FGF)
AAVALLPAVLLALLAP

SEQ ID NO: 70 - protein, 9 aa, Artificial Sequence
  Synthetic Sequence - N50 (NLS of NF-kB P50)
VQRKRQKLM

SEQ ID NO: 71 - protein, 21 aa, Artificial Sequence
  Synthetic Sequence - Pep-1
KETWWETWWTEWSQPKKKRKV

SEQ ID NO: 72 - protein, 15 aa, Artificial Sequence
  Synthetic Sequence - Pep-7
SDLWEMMMVSLACQY

SEQ ID NO: 73 - protein, 7 aa, Artificial Sequence
  Synthetic Sequence - Short Penetrating
RRMKWKK

SEQ ID NO: 74 - protein, 28 aa, Artificial Sequence
  Synthetic Sequence - Prion mouse PrPc1-28
MANLGYWLLALFVTMWTDVGLCKKRPKP

SEQ ID NO: 75 - protein, 18 aa, Artificial Sequence
  Synthetic Sequence - pVEC
LLIILRRRIRKQAHAHSK

SEQ ID NO: 76 - protein, 18 aa, Artificial Sequence
  Synthetic Sequence - SAP
VRLPPPVRLPPPVRLPPP

SEQ ID NO: 77 - protein, 7 aa, Artificial Sequence
  Synthetic Sequence - SV-40 (NLS)
PKKKRKV

SEQ ID NO: 78 - protein, 27 aa, Artificial Sequence
  Synthetic Sequence - Transportan
GWTLNSAGYLLGKINLKALAALAKKIL

SEQ ID NO: 79 - protein, 21 aa, Artificial Sequence
  Synthetic Sequence - Transportan 10
AGYLLGKINLKALAALAKKIL

SEQ ID NO: 80 - protein, 12 aa, Artificial Sequence
  Synthetic Sequence - Transportan derivative 1
GWTLNSAGYLLG

SEQ ID NO: 81 - protein, 34 aa, Artificial Sequence
  Synthetic Sequence - VP22
DAATATRGRSAASRPTERPRAPARSASRPRRPVD

SEQ ID NO: 82 - protein, 26 aa, Artificial Sequence
  Synthetic Sequence - VT5
DPKGDPKGVTVTVTVTVTGKGDPKPD

(19) United States
(12) Patent Application Publication
(10) Pub. No.: US 2022/0193016 A1
(43) Pub. Date: Jun. 23, 2022

(54) COMPOSITIONS, METHODS AND USES FOR TARGETING C-TERMINAL BINDING PROTEIN (CtBP) IN TRAUMATIC BRAIN INJURY

(71) Applicant: THE REGENTS OF THE UNIVERSITY OF COLORADO, A BODY CORPORATE, Denver, CO (US)

(72) Inventors: Mingxia HUANG, Greenwood Village, CO (US); David NORRIS, Greenwood Village, CO (US); Rui ZHAO, Englewood, CO (US)

(21) Appl. No.: 17/686,128

(22) Filed: Mar. 3, 2022

Related U.S. Application Data
(63) Continuation of application No. PCT/US2020/050145, filed on Sep. 10, 2020.
(60) Provisional application No. 62/899,047, filed on Sep. 11, 2019.

(51) Int. Cl. A61K 31/192; A61K 45/06; A61P 25/28

(52) U.S. Cl. CPC A61K 31/192; A61P 25/28; A61K 45/06

(57) ABSTRACT

Embodiments of the instant disclosure generally concern compositions, preparation and methods of use for inhibitory compounds of C-terminal binding proteins (CtBPs), for example, CtBP1 and CtBP2. Certain embodiments concern administering inhibitory compounds of CtBP1 and/or CtBP2 to a subject to treat or reduce the effects of a brain injury or brain trauma in a subject. In other embodiments, agents for inhibiting CtBP1 and/or CtBP2 to treat a subject having a traumatic brain injury (TBI) can include, but are not limited to a peptide capable of inhibiting CtBP expression and/or activity. In other embodiments, agents for inhibiting CtBP1 and/or CtBP2 to treat a subject having a TBI can include, but are not limited to, E1A peptide, a small molecule, an siRNA or a combination thereof. In certain embodiments, CtBP inhibitory agents can be administered alone or as a combination with other CtBP inhibitors.

### PRIORITY

This Continuation application claims priority to Patent Cooperation Treaty (PCT) Application No. PCT/US20/50145 filed Sep. 10, 2020, which PCT application claims priority to U.S. Provisional Patent Application No. 62/899,047 filed Sep. 11, 2019. Each of these applications is incorporated herein by reference in their entirety for all purposes.

### SEQUENCE LISTING

The instant application contains a Sequence Listing submitted electronically in ASCII format on Sep. 10, 2020 and corrected on Mar. 9, 2022 and the Sequence Listings are hereby incorporated by reference in their entirety for all purposes. The ASCII copy containing the same sequence information as originally filed, correcting a minor error was created on Mar. 9, 2022, is referenced as 106549-718910_CU4892H-US1_Sequence_listing.txt and is 24 Kilobytes in size.

### FIELD

Embodiments of the instant disclosure generally concern compositions, preparation and application of inhibitory compounds of the C-terminal binding protein (CtBP) 1 and CtBP2. Certain embodiments concern administering inhibitory compounds of CtBP1 and/or CtBP2 to a subject to treat or reduce the effects of a brain injury or brain trauma in a subject. In other embodiments, agents for inhibiting CtBP1 and/or CtBP2 to treat a subject having a traumatic brain injury can include, but are not limited to a peptide capable of inhibiting CtBP expression and/or activity. In other embodiments, agents for inhibiting CtBP1 and/or CtBP2 to treat a subject having a traumatic brain injury can include, but are not limited to, E1A peptide, a small molecule, an siRNA, a combination thereof or the like. In some embodiments, compositions disclosed herein can be administered to a subject to reduce or eliminate induction of CtBP-controlled inflammatory genes in the brain and/or circulating leukocytes, for example, to alleviate neurological deficits following traumatic brain injury (TBI). In certain embodiments, CtBP inhibitory agents can be administered alone or in combination with other TBI alleviating agents to reduce adverse effects of these molecules.

### BACKGROUND

Traumatic brain injury “TBI” can occur when an external force injures the brain, resulting in physical, cognitive, social, emotional, and/or behavioral impairment of a subject sustaining such an injury. In some cases, these impairments are temporary, but in other cases they can be prolonged and even permanent. The initial impact to a brain can result in primary damage that can be referred to as mild, moderate or severe TBI depending on the level of injury. Additional adverse intracranial and systemic effects, often termed “secondary injury” can occur after a TBI. A secondary injury can result in multiple additional biochemical, metabolic and cellular changes that occur following the initial injury. These changes can include systemic and brain inflammation, activation of microglia and astrocytes, changes in blood-brain barrier permeability, free radical overload, release of inflammatory factors, neurotransmitter release, absorption of calcium and sodium into neurons, changes in blood flow to the brain, ischemia, cerebral hypoxia, increased intracranial pressure, and mitochondrial dysfunction or combinations of these changes.

Mild TBI (mTBI) accounts for approximately 75% of the total TBI cases and has become a significant public health problem worldwide and especially in the United States. mTBI is termed a “silent epidemic” because many patients carry no outward physical signs of the illness and current diagnostic tests are often not sensitive or specific enough to identify individuals who have suffered a mTBI. No comprehensive treatment is currently available, in part because of the complexity of the problem, and its heterogeneity. Some mTBI patients sustain chronic changes in the brain white matter with long-term physical, cognitive, social, emotional, and behavioral impairment. Currently, very few treatments exist for traumatic brain injury to reduce the symptoms and improve recovery of the brain.

### SUMMARY

Certain embodiments disclosed herein concern compositions, methods for preparing and methods for treating, ameliorating and/or impeding effects of a traumatic brain injury (TBI) in a subject. In some embodiments, the TBI can be at least one of a diffuse brain injury (e.g. a concussion or concussion-like injury) and a focal brain injury. In other embodiments, compositions disclosed herein can be used for treating, ameliorating and/or impeding effects of the progression of the secondary injury in TBI. In accordance with these embodiments, compositions such as pharmaceutically acceptable compositions can include agents capable of inhibiting expression, translation and/or activity of C-terminal binding protein (CtBP) or proteins. In certain embodiments, CtBPs can include at least one of CtBP1 and CtBP2. In some embodiments, compositions can be administered to a subject for inhibiting expression, translation and/or activity of CtBPs and/or for inhibiting expression, translation and/or activity of genes controlled or activated by CtBPs (referred herein as “CtBP target genes”) in a subject experiencing TBI.

In some embodiments, compositions and methods disclosed herein can be used to target at least one of CtBP1 and CtBP2. CtBP1 and CtBP2 are transcriptional co-regulators that are recruited to their target gene promoters by association with various DNA-binding transcription factors. In certain embodiments disclosed herein, compositions for inhibiting expression, translation and/or activity of CtBPs (e.g. CtBP1 and CtBP2) can be used to treat TBI in a subject, for example by reducing mild TBI (mTBI) neuroinflammation including microglia and astrocyte activation or other CtBP activity in the subject and treating the TBI.

In certain embodiments, a CtBP inhibitor can be a peptide capable of inhibiting CtBP expression, translation and/or activity in order to treat TBI in a subject. In accordance with these embodiments, the peptide can be a peptide capable of disrupting a CtBP protein from activating downstream transcription factors or other molecules containing a CtBP-binding motif. In certain embodiments, an E1A protein or E1A polypeptide derived therefrom can be used to disrupt CtBP from binding a CtBP-binding motif and reduce or eliminate activation of downstream factors, for example chromatin remodeling factors (e.g. histone acetyltransferase (HAT), p300/CBP, histone deacetylase (HDAC), histone demethylases). In some embodiments, a disruptive peptide in these cascades can be used to reduce TBI effects, for example, reducing inflammatory responses from a TBI or a mTBI or a repetitive TBI or other type of TBI. In some embodiments, factors containing a CtBP-binding motif activated by CtBP1 and/or CtBP2 can be blocked by introducing an E1A protein or polypeptide thereof. In certain embodiments, the E1A protein or polypeptide can be conjugated to a cell penetrating peptide (CPP), for example, to enhance crossing into the blood brain barrier of the subject or increase the half-life of the peptide or direct the peptide to a targeted region of the brain of a subject. In some embodiments, a CPP can be Pep1, Tat, pAntp, polyarginine molecule (e.g., Arg8, Arg9, Arg11), plsl or similar cell penetrating peptide or blood brain penetrating peptide capable of being conjugated or linked to a peptide for inhibiting at least one of CtBP1 and CtBP2 to bind to its binding motif (e.g. PXDLS, SEQ. ID. 11). In some embodiments, the peptide can be a Pep1-E1A peptide. In certain embodiments, the peptide can be a Tat-E1A peptide. In other embodiments, the peptide can be a pAntp-E1A peptide. In certain embodiments, the peptide can be a polyarginine molecule (e.g., Arg8, Arg9, Arg11)-E1A peptide. In certain embodiments, the peptide can be a dNP2 peptide. In yet other embodiments, the peptide can be a plsl-E1A peptide. In certain embodiments, the peptide can be derived from or a peptide fragment of, or a peptide fragment having biological activity of E1A, or a E1A peptide fragment having CtBP-binding motif activity. In other embodiments, the E1A's CtBP-binding motif can be a polypeptide including a PXDLS-containing fragment (X is any amino acid or any hydrophobic amino acid) or PXDLS (SEQ ID NO. 11) or PX1DLSX2K (X1 is any amino acid or any hydrophobic amino acid; X2 is any amino acid) (SEQ ID NO. 12) or a E1A mutant thereof containing the ability to bind the CtBP-binding motif. In some embodiments, the E1A's CtBP-binding motif is a polypeptide including PXDLS (SEQ ID NO. 11) or PX1DLSX2K (SEQ ID NO. 12) or a E1A mutant thereof containing binding properties to a CtBP-binding motif.

In certain embodiments, the CtBP1 and/or CtBP2 or other CtBP inhibitor can be a small interfering RNA (“si”) that targets CtBP1 “siCtBP1” or CtBP2 “siCtBP2”. In other embodiments, the CtBP inhibitor can be a small molecule. In certain embodiments, the small molecule may be NSC95397 or equivalent molecule thereof having CtBP-binding motif inhibitory properties. In some embodiments, a small interfering RNA can be provided to a subject having a TBI in order to reduce side effects of a TBI; for example, in the place of an E1A polypeptide referenced above. In other embodiments, a small interfering RNA can be provided to a subject having a TBI in order to enhance effects of any other inhibitory agent contemplated herein such as a E1A polypeptide, small molecule or other agent contemplated herein for administration as a combination of agents, co-administered or sequentially administered to a subject.

In some embodiments, a small molecule for inhibiting or blocking or reducing a CtBP activity can be 2-Oxo-4-methylthiobutanoic acid “MTOB” or its derivatives thereof. In accordance with these embodiments, MTOB or its derivatives thereof reduce, inhibit or block CtBPs dehydrogenase activity, reduces or inhibits CtBP's transcriptional co-regulatory activity or a combination thereof. In accordance with these embodiments, a MTOB derivative can include, but is not limited to, phenylpyruvate, 2-hydroxyimino-3-phenylpropanoic acid (HIPP), 3-Cl-HIPP, 4-Cl-HIPP and 3-OH-HIPP or a combination thereof or other molecule similar thereto. It is contemplated herein that MTOB or a derivative thereof can be part of a combination of CtBP-binding motif inhibitory agents disclosed herein to reduce side effects of TBI in a subject. For example, a CtBP binding motif inhibitor (e.g. E1A polypeptide) and a CtBP dehydrogenase activity inhibitor (e.g. MTOB, HIPP) can be combined, simultaneously administered, sequentially administered or alternatively administered to a subject to treat a TBI in a subject.

In certain embodiments, the TBI can be a mild TBI, a moderate TBI, or a severe TBI. In some embodiments, the TBI can occur in a healthy subject. In other embodiments, the TBI can occur in a subject suffering from another cognitive or memory condition; for example, Alzheimer's or other similar condition affecting the hippocampus of the brain or other region having CtBP-induced inflammation. In yet other embodiments, the subject can be suffering from a mental disorder affecting the cerebral cortex or hippocampus of the brain of a subject affecting memory, cognition and emotion and other functions and sustain a TBI that can be treated by compositions or combinations compositions disclosed herein. In certain embodiments, TBI can include a diffuse injury including, but not limited to diffuse axon injury such as a concussion or whiplash from an accident or injury (e.g. sports-related TBI). In other embodiments, the compositions disclosed herein can be used to stabilize, ameliorate and/or restore brain activities in a treated subject to restore these functions to their pre-TBI state.

In certain embodiments, agents disclosed herein capable of reducing, inhibiting or eliminating CtBP-related activities can be administered during an acute phase of the TBI, during a subacute phase of the TBI, or during a chronic phase of TBI in a subject. In some embodiments, administration of compositions disclosed herein to reduce, inhibit or eliminate CtBP binding to CtBP binding motifs and/or reduce CtBP dehydrogenase activity can be administered to a subject having had a TBI within: a week, 6-days, 5-days, 4-days, 3-days, 2-days, 1-day or on the day of experiencing a TBI. In other embodiments, administration of compositions disclosed herein to reduce, inhibit or eliminate CtBP binding to CtBP binding motifs and/or reduce CtBP dehydrogenase activity can be administered to a subject having had a TBI in addition to another brain disorder (e.g. Alzheimer's) within: a week, 6-days, 5-days, 4-days, 3-days, 2-days, 1-day or on the day of experiencing a TBI. In other embodiments, compositions disclosed herein can be administered to a subject having a TBI to reduce inflammation in the brain and systemic inflammation. In some embodiments, the compositions disclosed herein can be administered twice daily, once daily, every other day, every third day or other regimen depending on responsiveness to the treatment and severity of the TBI condition as well as time from experiencing the TBI. In certain embodiments, a subject in the chronic phase after a TBI can be treated with compositions disclosed herein to improve memory, cognition, emotion and other functions of the brain in a subject.

In some embodiments, CtBP inhibitors disclosed herein can be used to treat TBI and TBI side effects. In some embodiments, CtBP inhibitors disclosed herein can be used to reduce neuroinflammation and systemic inflammation of a subject having a TBI, for example, reducing expression of pro-inflammatory cytokines, cell adhesion molecules involved in leukocyte recruitment, alarmins and inflammasome components, promoting neuronal survival and proliferation of glial cells for repair and recovery of function.

### Definitions

As used herein, the singular forms “a,” “an,” and “the” encompass implementations having plural referents, unless the content clearly dictates otherwise. As used in this specification and the appended claims, the term “or” is generally employed in its sense including “and/or” unless the content clearly dictates otherwise.

As used herein, “inhibit,” “inhibition” and “inhibiting” can include any method known in the art or described herein, which results in measurable reduction in the expression or function of one or more CtBP or downstream factor. As used herein, inhibiting can include reducing, eliminating, preventing any relative decrease function or production of a gene product, up to and including complete elimination of production or function of the gene product or factor or activity of the targeted agent to be inhibited. Inhibition can be measured by any suitable method known in the art, including, but not limited to, methods used in the Examples below, for example, comparison of mRNA transcript levels, protein or peptide levels, and/or phenotypes including cytokine production and secretion levels, inflammatory marker analysis, cellular morphology changes, and neurobehavioral test scores.

As used herein, the term “polynucleotide” can refer to a sequence of covalently-linked nucleotides in which the 3′ position of a pentose of one nucleotide is linked to the 5′ position of a pentose of the following nucleotide by a phosphodiester group. Nucleotides can include deoxyribonucleotides (DNA) or ribonucleotides (RNA). Polynucleotide can refer to either DNA or RNA. In some embodiments, a polynucleotide can be a messenger RNA (mRNA).

As used herein, “peptide” can refer to a chain of two or more amino acids bonded together, typically with the carboxyl group of each acid linked to the amino group of the next; and “polypeptide” refers generally to a linear organic polymer including a plurality of amino acid residues bonded together, forming at least a part of a protein molecule. The terms “polypeptide” or “peptide” can include polypeptides or peptides with or without any modifications including for example, post-translational modifications.

As used herein, the term “expression” can refer to gene transcription and translation. For example, the term “decreases the expression” can mean a reduction in gene transcription and translation or one or the other as measured by observation of levels of a gene product such as an RNA or a polypeptide, or a phenotype related to such expression.

As used herein, “C-terminal binding protein,” “CtBP1,” “CtBP2,” and “CtBP” can refer to a transcriptional regulator which binds to a C-terminus of E1A proteins.

As used herein, “E1A peptide” can refer to the E1A oncoprotein or fragment of E1A thereof.

As used herein, “treating or ameliorating traumatic brain injury” can include alleviating symptoms or signs of the TBI.

As used herein, “effective amount” can refer to an amount of a substance, such as a therapeutic substance, sufficient to achieve an intended purpose or effect. Many factors may affect the ability of a substance to perform its intended task when administered to a subject. One of skill in the art would understand that an “effective amount” can depend upon such factors, including biological factors.

As used herein, a “therapeutically effective amount” can refer to an amount of a substance which is capable of achieving a desired physiologic or psychologic result to a selected degree. While the achievement of therapeutic effects can be measured by health provider using evaluations known in the art

As used herein, “RNA interference” (“RNAi”) is a method of gene regulation conducted post-transcriptionally, which is conserved in many eukaryotic organisms. RNAi is induced by short double-stranded RNA nucleotides (typically less than about 30 nucleotides), “dsRNA” which are present in the cell. These dsRNA molecules, often referred to as “short interfering RNA” or “siRNA,” trigger destruction of mRNAs which share sequence homology with the siRNA. In some cases, this shared homology is within one nucleotide resolution. Without being limited to any one theory, it is believed that the siRNA and the targeted mRNA bind to a complex which cleaves the mRNA, referred to as an RNA-induced silencing complex (“RISC”). siRNAs appear to be reused within a cell, with each being capable of inducing cleavage of around 1000 mRNA molecules. Those skilled in the art regard siRNA-mediated RNAi as highly effective for inhibiting expression of a target gene, such as, in this instance, CtBP.

Synthetic siRNA has been demonstrated to induce RNAi of target mRNA. siRNA-mediated RNAi has been shown to have potential therapeutic use by several studies. RNAi demonstrated potential for use in human cancer cells as well. As used herein, “siCtBP1” and/or “siCtBP2” can refer to a class of CtBP-specific siRNAs, for example, siRNAs sharing homology with CtBP mRNAs which trigger destruction of the CtBP mRNAs when administered to a cell or organism expressing CtBP.

As used herein, a CtBP inhibitor can include any molecule which causes measurable reduction in the expression or function or activity of one or more CtBP. In certain embodiments, CtBP inhibitors can include, but are not limited to, compounds which, when introduced to a system such as a cell, result in a relative decrease in function, activity or production of CtBP, up to and including complete elimination of production, activity or function of CtBP or targeted factors downstream of CtBP proteins. Inhibitors of CtBP contemplated herein can include, but are not limited to, siRNAs (siCtBP1, siCtBP2), peptide inhibitors and small-molecule inhibitors. Peptide inhibitors can include, but are not limited to, E1A peptides, for example, cell penetrating peptide “CPP”-E1A peptides. In other embodiments, CPP-E1A peptides (cell penetrating peptide E1A) can include, but are not limited to Pep1-E1A, Tat-E1A, pAntp-E1A, dNP2-E1A, Arg9-E1A, plsl-E1A, and other inhibitory peptides derived from E1A. Other CtBP inhibitors contemplated herein can include, but are not limited to, small molecule inhibitors which can include, but are not limited to, NSC95397, MTOB, phenyl pyruvate, HIPP, 3-Cl-HIPP, 4-Cl-HIPP, 3-OH-HIPP and derivatives thereof.

As used herein, “administration,” and “administering” can be used interchangeably. Each refers to a method of treating by presenting, applying, or introducing an agent such as a CtBP inhibitor or more than one CtBP inhibitor or modifying agent to a subject in order to achieve a desired physiological response. Such agents can be formulated for administration and provided as a “formulation.” As used herein, “formulation” can be a composition for providing an agent, optionally employing pharmaceutically acceptable carriers, as known in the art, to a subject, using any suitable method of delivery, e.g., oral, sublingual, intravenous, subcutaneous, transcutaneous, intramuscular, intracutaneous, intrathecal, epidural, intraocular, intracranial, inhalation, rectal, vaginal, and the like administration. Administration in the form of creams, lotions, tablets, capsules, pellets, dispersible powders, granules, suppositories, syrups, elixirs, lozenges, injectable solutions, sterile aqueous or non-aqueous solutions, suspensions or emulsions, patches, and the like, is also contemplated. Active agents can be compounded with non-toxic, pharmaceutically acceptable carriers including, but not limited to, glucose, lactose, gum acacia, gelatin, mannitol, starch paste, magnesium trisilicate, talc, corn starch, keratin, colloidal silica, potato starch, urea, dextrans, and the like.

In the following description, reference is made to the accompanying drawing that forms a part hereof and in which are shown by way of illustration at least one specific implementation. The following description provides additional implementations. It is to be understood that other implementations are contemplated and may be made without departing from the scope or spirit of the present invention.

### BRIEF DESCRIPTION OF THE DRAWINGS

FIG. 1A represents an illustration of multifaceted roles of CtBP-mediated responses in traumatic brain injury (TBI) and representative inhibitory molecules of some embodiments disclosed herein.

FIG. 1B illustrates a schematic representation of domain structure of the CtBP proteins and structures of three small-molecule inhibitors of CtBP of some embodiments disclosed herein.

FIGS. 2A-2C illustrate CtBP-dependent induction of inflammatory genes in lipopolysaccharide (LPS)-activated microglia and macrophage cell lines, A) BV2 cells; B) RAW264.7 cells and c) a histogram plot of promoter binding of CtBP of some embodiments disclosed herein.

FIGS. 3A-3F illustrate examples of dose responses and time courses of expression of mRNAs and proteins for CtBP2 and CtBP target genes in the Closed Head Impact Model of Engineered Rotational Acceleration (CHIMERA) mouse model of mTBI in response to a single impact at the input energy range of 0.5-0.8 J, A) Dose effect in brain; B) Dose effect in blood; C) Dose effect in brain (Western Blot); D) Time course in brain; E) Time course in blood; and F) Time course in brain (Western Blot) of some embodiments disclosed herein.

FIGS. 4A-4G illustrates immunohistochemistry images demonstrating TBI-induced microglia activation and induction of CtBP2 protein in the brain where A) represents a coronal section through the hippocampus and the corpus callosum; B-D) represent sham brain sections; E-G) represent TBI brain sections of some embodiments disclosed herein.

FIGS. 5A-5B. represent histogram plots of comparisons of expression of CtBP2 and CtBP target genes in peripheral blood leukocytes (B) and at local skin regions (A) near initial impact of the mouse model directly impacted by an energy dose of some embodiments disclosed herein.

FIGS. 6A-6C illustrate in 6A, a histogram plot of mRNA levels of various agents with and without various peptide inhibitors disclosed herein; 6B illustrates immunostaining images of treated versus untreated microglia; and 6C illustrates a histogram plot of a comparison of treated and/or induced isolated mouse hippocampus of some embodiments disclosed herein.

FIGS. 7A-7C represents in 7A a plot of Neurological Severity Score (NSS) scores at various time points post TBI; 7B and 7 C represent histogram plots of examples of mRNA expression of various genes in the brain and peripheral blood leukocytes of treated and untreated samples of some embodiments disclosed herein.

FIGS. 8A-8C represents in 8A a plot of NSS scores at various time points post TBI; 8B and 8C represent histogram plots of examples of mRNA expression of various genes in the brain and peripheral blood leukocytes of small molecule treated and untreated samples of some embodiments disclosed herein.

FIGS. 8D-8H illustrates exemplary data of a small molecule compound treated and untreated TBI-triggered activation of microglia and astrocytes in the brain in of some embodiments disclosed herein.

FIGS. 9A-9D represents in A, a time-course of the experiment; B represents a histogram plot comparison of Loss of Righting Reflex (LRR) durations; C represents NSS scores in the presence and absence of CtBP inhibitors; and D represents histogram plots of relative mRNA levels of certain genes in the presence or absence of CtBP inhibitors repetitive of mild TBI of some embodiments disclosed herein.

FIGS. 10A-10B illustrates an exemplary MTOB pre-treatment inhibiting expression of several CtBP target genes in LPS-activated A) mouse microglia cell line BV2 and B) mouse macrophage cell line RAW264.7 of some embodiments disclosed herein.

FIG. 11 illustrates an exemplary MTOB post-treatment inhibiting expression of several CtBP target genes in LPS-activated mouse microglia of some embodiments disclosed herein.

FIGS. 12A-12B illustrate CtBP2 expression assessed by immunohistochemistry (IHC) in the dentate gyrus region of the hippocampus of a transgenic rat modeling Alzheimer disease (AD) pathology (A) and in the cingulate cortex and dentate gyrus after receiving repeated Controlled Cortical Impact (CCI), a type of focal TBI (B) of some embodiments disclosed herein.

FIGS. 13A-13B illustrate use of two exemplary peptides in an inflammatory model of some embodiments disclosed herein.

FIGS. 14A-14B illustrate use of additional exemplary peptides compared to a control peptide and a negative control in another inflammatory model of some embodiments disclosed herein.

Incorporated herein by reference in its entirety for all purposes is the sequence listing entitled “Sequence Listing ST25” of 50 kilobytes in size.

### DETAILED DESCRIPTION

Certain embodiments disclosed herein concern compositions, methods for preparing and methods for treating, ameliorating and/or impeding effects of a traumatic brain injury (TBI) in a subject. In some embodiments, the TBI can be at least one of a diffuse brain injury (e.g. a concussion or concussion-like injury) and a focal brain injury. In other embodiments, compositions disclosed herein can be used for treating, ameliorating and/or impeding effects of the progression of the secondary injury in TBI. In accordance with these embodiments, compositions such as pharmaceutically acceptable compositions can include agents capable of inhibiting expression, translation and/or activity of C-terminal binding protein (CtBP) or proteins. In certain embodiments, CtBPs can include at least one of CtBP1 and CtBP2. In some embodiments, compositions can be administered to a subject for inhibiting expression, translation and/or activity of CtBPs and/or for inhibiting expression, translation and/or activity of genes controlled or activated by CtBPs (referred herein as “CtBP target genes”) in a subject experiencing TBI.

In some embodiments, compositions and methods disclosed herein can be used to target at least one of CtBP1 and CtBP2. CtBP1 and CtBP2 regulate gene transcription in diverse biological processes by directly binding DNA-binding transcription factors and recruiting chromatin-modifying enzymes to target gene promoters. In certain embodiments disclosed herein, compositions for inhibiting expression, translation and/or activity of CtBPs (e.g. CtBP1 and CtBP2) can be used to treat TBI in a subject, for example by reducing mild TBI (mTBI)-associated neuroinflammation and systemic inflammation or other CtBP activity in the subject and treating the TBI.

In certain embodiments, a CtBP inhibitor can be a peptide capable of inhibiting CtBP expression, translation and/or activity in order to treat TBI in a subject. In accordance with these embodiments, the peptide can be a peptide capable of disrupting a CtBP protein from cooperating with transcription factors or other molecules containing a CtBP-binding motif. In certain embodiments, an E1A protein or E1A polypeptide derived therefrom can be used to disrupt CtBP from binding a CtBP-binding motif and reduce or eliminate activation of downstream factors, for example chromatin remodeling factors (e.g. histone acetltransferase (HAT), p300/CBP, histone deacetylase (HDAC), histone demethylases). In some embodiments, a disruptive peptide in these cascades can be used to reduce TBI effects, for example, reducing inflammatory responses from a TBI or a mTBI or a secondary TBI or other type of TBI. In some embodiments, factors containing a CtBP-binding motif activated by CtBP1 and/or CtBP2 can be blocked by introducing an E1A protein or polypeptide thereof. In certain embodiments, the E1A protein or polypeptide can be conjugated to a cell penetrating peptide (CPP), for example, to enhance crossing into the blood brain barrier of the subject or increase the half-life of the peptide or direct the peptide to a targeted region of the brain of a subject. In some embodiments, a CPP can be Pep1, Tat, pAntp, polyarginine molecule (e.g., Arg8, Arg9, Arg11 or other suitable length Arg, Arg(X) where X can be from about 2 to about 25), dNP2, plsl or similar cell penetrating peptide or blood brain penetrating peptide capable of being conjugated or linked to a peptide for inhibiting at least one of CtBP1 and CtBP2 to bind to its binding motif (e.g. PXDLS). In some embodiments, the peptide can be a Pep1-E1A peptide. In certain embodiments, the peptide can be a Tat-E1A peptide. In other embodiments, the peptide can be a pAntp-E1A peptide. In certain embodiments, the peptide can be a polyarginine molecule (e.g., Arg8, Arg9, Arg11)-E1A peptide and can be with myristoylation of the polyarginine backbone to enhance permeability through the blood-brain barrier. In other embodiments, the peptide can be a dNP2-E1A (dNP2 is a blood-brain barrier-permeable peptide). In yet other embodiments, the peptide can be a plsl-E1A peptide. In certain embodiments, the peptide can be derived from or a peptide fragment of, or a peptide fragment having biological activity of E1A, or a E1A peptide fragment having CtBP-binding motif activity. In other embodiments, the E1A's CtBP-binding motif can be a polypeptide including a PXDLS-containing fragment (X is any amino acid or any hydrophobic amino acid) or PXDLS (SEQ. ID. NO. 11) or PXDLSX2KK (SEQ ID. NO: 12) or a E1A mutant thereof containing the ability to bind the CtBP-binding motif. In some embodiments, the E1A's CtBP-binding motif is a polypeptide including PXDLS (SEQ. ID. NO. 11) or PXDLSX2KK (SEQ ID. NO: 12) or a E1A mutant thereof containing binding properties to a CtBP-binding motif. In some embodiments, peptide sequences of use in compositions and methods disclosed herein can be represented by those of Table 1 inserted below.

TABLE 1
Exemplary Peptide Sequences

| Name | SEQ ID No. | Amino acid Sequence (N-terminus to C-terminus) |
|---|---|---|
| Pep1- | 5 | GSHMKETWWETWWTEWSQPKKKRKVLEEPGQPL |
| E1A_{WT} | | DLSCKRPRDYKDDDDK |
| Pep1- | 6 | GSHMKETWWETWWTEWSQPKKKRKVLEEPGQPL |
| E1A_{Mut} | | DELCKRPRDYKDDDDK |
| Tat- | 7 | GRKKRRQRRRPPQLEEPGQPLDLSCKRPR |
| E1A | | |
| E1A-5 | 11 | PXDLS (X is any amino acid) |
| E1A-7 | 12 | PX1DLSX2K (X1 and X2 are as defined herein, any amino acid) |
| E1A- | 13 | EQTVPVDLSVARPR |
| 14eq | | |
| E1A- | 14 | GGDGPLDLCCRKRP |
| 14gg | | |
| E1A- | 15 | PTDEPLNLSLKRPR |
| 14pt | | |
| E1A- | 16 | EPGQPLDLSCKRPR |
| 14ep | | |
| MH-1 | 18 | RRWRRWNRFNRRRGGPIDLSKKA |
| MH-2 | 19 | RRRRRRRRGGPIDLSKKA |
| MH-3 | 20 | RRRRRRRRPIDLS |
| MH-4 | 21 | RRRRRGGPIDLSKK |
| MH-6 | 22 | RRRRRRRRPIDLSKKA |
| MH-7 | 23 | RRRRRRRRGGPIDLSKK |

In certain embodiments, the CtBP1 and/or CtBP2 or other CtBP inhibitor can be a small interfering RNA (“si”) that targets CtBP1 “siCtBP1” or CtBP2 “siCtBP2”. In certain embodiments, siRNA sequences of use herein can be represented by the sequences of Table 2 below.

TABLE 2
Exemplary siRNA sequences.

| Name | SEQ ID No. | RNA Sequence (5′ to 3′) |
|---|---|---|
| siCtBP 1 | 1 | UCUUCCACAGUGUGACUGCGUUAUUUU |
| (mouse) | | |
| siCtBP2 | 2 | GCCUUUGGAUUCAGCGUCAUAUUU |
| (mouse) | | |
| siCtBP 1 | 3 | ACGACUUCACCGUCAAGCAUU |
| (human) | | |
| siCtBP2 | 4 | GCGCCUUGGUCAGUAAUAGdTdT |
| (human) | | |

In other embodiments, the CtBP inhibitor can be a small molecule. In certain embodiments, the small molecule may be NSC95397 or equivalent molecule thereof having CtBP-binding motif inhibitory properties. In some embodiments, a small interfering RNA can be provided to a subject having a TBI in order to reduce side effects of a TBI; for example, in the place of an EIA polypeptide referenced above. In other embodiments, a small interfering RNA can be provided to a subject having a TBI in order to enhance effects of any other inhibitory agent contemplated herein such as a E1A polypeptide, small molecule or other agent contemplated herein for administration as a combination of agents, co-administered or sequentially administered to a subject.

In some embodiments, a small molecule for inhibiting or blocking or reducing a CtBP activity can be 2-Oxo-4-methylthiobutanoic acid “MTOB” or its derivatives thereof. In accordance with these embodiments, MTOB or its derivatives thereof reduce, inhibit or block CtBPs dehydrogenase activity, reduces or inhibits CtBP's transcriptional co-regulatory activity or a combination thereof. In accordance with these embodiments, a MTOB derivative can include, but is not limited to, phenylpyruvate, 2-hydroxyimino-3-phenylpropanoic acid (HIPP), 3-Cl-HIPP, 4-Cl-HIPP and 3-OH-HIPP or a combination thereof or other molecule similar thereto. It is contemplated herein that MTOB or a derivative thereof can be part of a combination of CtBP-binding motif inhibitory agents disclosed herein to reduce side effects of TBI in a subject. For example, a CtBP binding motif inhibitor (e.g. E1A polypeptide) and a CtBP dehydrogenase activity inhibitor (e.g. MTOB, HIPP) can be combined, simultaneously administered, sequentially administered or alternatively administered to a subject on the same or different days or schedules to treat a TBI in a subject.

In certain embodiments, some CtBP inhibitors can be a small molecule. In certain embodiments, the small molecule can be NSC95397, MTOB, phenylpyruvate, 2-hydroxyimino3-phenylpropanoic acid (HIPP) or an analog or derivative thereof. In other methods, some of the small molecules can be 3-Cl-HIPP, 4-Cl-HIPP, 3-OH-HIPP, 4-Me HIPP, 3-Me HIPP, 2-Me HIPP, 4-OMe HIPP, 3-OMe HIPP, 2-OMe HIPP, 4-Cl HIPP, 3-Cl HIPP, 2-Cl HIPP, 4-OH HIPP, 3-OH HIPP, 4-F HIPP, 4-CN HIPP, or any combination thereof.

HIPP is a member of the D2-HDH-based class 2 of CtBP-specific inhibitors with the formula 2-hydroxyimino-3-phenylpropanoic acid (HIPP). References to HIPP can also include its analogs/derivatives including, but not limited to, 3-Cl-HIPP, 4-Cl-HIPP and 3-OH-HIPP. Other HIPP derivatives have been reported, including, but not limited to, derivatives with various substitutions on the phenyl ring of HIPP, including 4-Me, 3-Me, 2Me, 4-OMe, 3-OMe, 2-OMe, 4-Cl, 3-Cl, 2-Cl, 4-OH, 3-OH, 4-F, and 4-CN substitutions. These compounds are at least substrate-competitive inhibitors of CtBP's dehydrogenase activity and interfere with its transcriptional co-regulatory function and of use to inhibit or prevent downstream CtBP gene activation and/or dehydrogenase activities of CtBPs. Other mechanisms of these agents to reduce side effects of CtBP are not yet known.

In certain embodiments, the TBI can be a mild TBI, a moderate TBI, or a severe TBI. In some embodiments, the TBI can occur in a healthy subject. In other embodiments, the TBI can occur in a subject suffering from another cognitive or memory condition; for example, Alzheimer's or other similar condition affecting the hippocampus of the brain or other region having CtBP-induced inflammation. In yet other embodiments, the subject can be suffering from a mental disorder affecting the cerebral cortex or hippocampus of the brain of a subject affecting memory, cognition and emotion and other functions. In certain embodiments, TBI can include a diffuse injury including, but not limited to diffuse axon injury such as a concussion or whiplash from an accident or injury (e.g. sports-related TBI). In certain embodiments, a subject can include a subject having or developing another brain condition or disease. In accordance with these embodiments, a subject can be a subject having Alzheimer's or other cognitive diminishing condition or disease. In other embodiments, compositions disclosed herein can be used to stabilize, ameliorate and/or restore brain activities in a treated subject to restore these functions in certain cases to their pre-TBI state or near pre-TBI state.

In certain embodiments, agents disclosed herein capable of reducing, inhibiting or eliminating CtBP-related activities can be administered during an acute phase of the TBI, during a subacute phase of the TBI, or during a chronic phase of TBI in a subject. In some embodiments, administration of compositions disclosed herein to reduce, inhibit or eliminate CtBP binding to CtBP binding motifs and/or reduce CtBP dehydrogenase activity can be administered to a subject having had a TBI within: a week, 6-days, 5-days, 4-days, 3-days, 2-days, 1-day or on the day of experiencing a TBI, for example within hours of sustaining a TBI. In other embodiments, administration of compositions disclosed herein to reduce, inhibit or eliminate CtBP binding to CtBP binding motifs and/or reduce CtBP dehydrogenase activity can be administered to a subject having had a TBI in addition to another brain disorder (e.g. Alzheimer's) within: a week, 6-days, 5-days, 4-days, 3-days, 2-days, 1-day or on the day of experiencing a TBI, for example within hours of sustaining a TBI. In other embodiments, compositions disclosed herein can be administered to a subject having a TBI to reduce inflammation in the brain. In some embodiments, the compositions disclosed herein can be administered twice daily, once daily, every other day, every third day or other regimen depending on responsiveness to the treatment and severity of the TBI condition as well as time from experiencing the TBI, age of the subject and other considerations generally applied by a health professional. In certain embodiments, a chronic TBI subject can be treated with compositions disclosed herein to improve memory, cognition, emotion and other functions of the brain in a subject.

In some embodiments, CtBP inhibitors disclosed herein can be used to treat TBI and TBI side effects. In some embodiments, CtBP inhibitors disclosed herein can be used to reduce neuroinflammation in the brain of a subject having a TBI or had a TBI, for example, by reducing expression of or induction of pro-inflammatory cytokines, cell adhesion molecules involved in leukocyte recruitment, alarmins and inflammasome components, promoting neuronal survival and proliferation of glial cells for repair and recovery of function.

TBI can be classified depending on the severity of the injury. In certain embodiments, mild TBI is contemplated. Mild TBI (mTBI) can generally refer to injuries resulting in loss of consciousness and/or disorientation is about 30 minutes or less. For those experiencing mTBI, medical imaging is often inadequate to identify abnormalities. Over time, a subject having a mTBI can experience side effects of the TBI including, but not limited to, headache, difficulty thinking, memory problems, attention deficits, mood swings and frustration, fatigue, visual disturbance, sleep disturbances, dizziness/loss of balance, irritability/emotional disturbances, feelings of depression, seizures, nausea, loss of smell, and/or sensitivity to light and sounds. In certain embodiment, initial injury to the brain in mTBI can trigger a cascade of delayed secondary injury responses due at least in part to biochemical changes. In some embodiments contemplated herein, these secondary brain injury periods can create a window for therapeutic intervention to reduce or even prevent progressive tissue and other damage.

In other embodiments, moderate traumatic brain injury generally refers to a brain injury resulting in a loss of consciousness of the subject from about 30 minutes to about 6 hours. In yet other embodiments, severe traumatic brain injury generally refers to a brain injury resulting in a loss of consciousness of greater than about 6 hours. A person experiencing moderate or severe traumatic brain injury can experience side effects including, but not limited to, receptive aphasia; expressive aphasia; slurred speech; reading problems; writing problems; difficulties interpreting touch, temperature sensitivities and fluctuations, issues with movement, issues with limb position and fine motor discrimination; partial or total loss of vision; weakness of eye muscles; double vision; blurred vision; involuntary eye movements; intolerance to light; hearing loss; ringing in the ears; sensitivity to sounds; anosmia; diminished sense of taste; seizures; physical paralysis; chronic pain; sleep disorders; emotional challenges; lack of motivation; irritability; aggression; depression; other emotional and mental issues; and lack of awareness or combinations thereof.

In certain embodiments, severity of a TBI can be assessed based on industry standards and can be used to evaluate treatments disclosed herein, before, during or after experiencing a TBI. For example, TBI can be assessed based on the Glasgow Coma Scale that considers multiple factors when determining the level of severity of a TBI. The factors can include, but are not limited to, a subject's motor response, verbal response, and eye response. A value of 13 to 15 on the Glasgow Coma Scale generally relates to mild traumatic brain injury. A value of 9-12 on the Glasgow Coma Scale generally relates to moderate traumatic brain injury. A value of 3-8 on the Glasgow Coma Scale generally relates to severe traumatic brain injury.

In certain embodiments, an acute TBI can include an injury that has occurred within approximately the previous three months. In some embodiments, the acute phase of an acute traumatic brain injury can further include a subacute phase which can occur between about six weeks and about three months following the brain injury. In other embodiments, a chronic phase TBI can include a TBI that occurred in a period of over three months since the occurrence.

In some embodiments, compositions disclosed herein can be administered to subject having a TBI. In certain embodiments, the compositions disclosed herein can be used to treat neuroinflammation caused by mild, moderate to severe TBIs, a factor in secondary injury from a TBI. In some embodiments, inhibition of agents capable of inducing neuroinflammation can be used to modulate the neuroinflammatory response in subjects experiencing traumatic brain injury.

In some embodiments, small-molecule compounds such as MTOB can be used as a substrate inhibitor of CtBP dehydrogenase activity. MTOB or similar small molecule compounds can be used to displace CtBPs from their target gene promoters and reduce dehydrogenase activity and inhibit or reduce induction of CtBP target genes. MTOB or similar small molecule compounds can be used to inhibit or block cytoplasm-to-nucleus translocation of NFκB, a transcriptional factor known for inducing inflammatory responses. In some embodiments, neuroinflammation is considered a secondary injury mechanism of TBI and can be manipulated through administration of therapeutics disclosed herein to reduce neuroinflammation and its side effects. In addition, CtBP 1 and CtBP 2 mediate transactivation of proinflammatory cytokines and other proinflammatory molecules including, but not limited to, IL1B, IL6 and TNFα during inflammatory responses in microglia, astrocytes and macrophages. Peptides, small molecules, and other types of compounds as disclosed herein can be used to disrupt interactions of CtBP activities; for example, to disrupt CtBP interactions with DNA-binding transcription factor partners and/or between CtBP proteins, which recruit CtBPs to specific target promoters and recruit additional proteins involved in chromatin remodeling.

In certain embodiments, the inhibitory peptide comprises no more than about 25 amino acids. In certain embodiments, the inhibitory peptide comprises no more than about 15 amino acids. In certain embodiments, the peptide construct is modified for conjugation to a carrier molecule. In certain embodiments, the cell penetrating peptide is an amphipathic peptide or anionic peptide. In certain embodiments, the cell penetrating peptide is a cationic peptide. In certain embodiments, the cell penetrating peptide is selected from the group consisting of Tat, pAntp, Arg9, plsl, and Pep1. In certain embodiments, the cell penetrating peptide is directly fused to the inhibitory peptide. In certain embodiments, the cell penetrating peptide is fused to the inhibitory peptide via a peptide linker. In certain embodiments, the cell penetrating peptide is fused to the N-terminus of the inhibitory peptide. In some embodiments, cell penetrating peptides can be represented by those illustrated in Table 3 below.

TABLE 3
Exemplary Cell Penetrating Peptides

| SEQ ID | Cell Penetrating | Amino acid Sequence |
|---|---|---|
| NO. | Peptide Designation | (N-terminus to C-terminus) |
| 8 | Penetracin (pAntp₄₃₋₅₈) | RQIKIWFQNRRIVIKWKK |
| 9 | Polyarginine-Rn (n = | Rn (n = 2-25) |
| | 5-20) | |
| 10 | Pls1 (Igl1 | RVIRVWFQNKRCKDKK |
| | homeodomain | |
| 17 | dNP2 | KIKKVKKKGRKKIKKVKKKGRK |
| 24 | TAT₄₇₋₅₇ | YGRKKRRQRRR |
| 25 | Tat₄₇₋₆₀ | YGRKKRRQRRRPPQ |
| 26 | Tat₄₈₋₆₀ | GRKKRRQRRRPPQ |
| 27 | Tat₄₈₋₆₁ | GRKKRRQRRRPPQQ |
| 28 | Tat₄₉₋₅₇ | RKKRRQRRR |
| 29 | SynB1 | RGGRLSYSRRRFSTSTGR |
| 30 | SynB3 | RRLSYSRRRF |
| 31 | SynB4 | AWSFRVSYRGISYRRSR |
| 32 | SynB5 | TGGRLAYLRRRWAVLGR |
| 33 | Angiopep-2 | PFFYGGSGGNRNNYLREEY |
| 34 | Angiopep-5 | RFFYGGSRGKRNNFRTEEY |
| 35 | FGF4 | AAVLLPVLLAAP |
| 36 | RDP | KSVRTWNEIIPSKGCLRVGGRCHPHVNGGGRRRRRRRRR |
| 37 | TAT-HA | YGRKKRRQRRR-YPYDVPDVA |
| 38 | ARF (1-22) | MVRRFLVTLRIRRACGPPRVRV |
| 39 | BPrPr (1-28) | MVKSKIGSWILVLFVAMWSDVGLCKKRP |
| 40 | P28 | LSTAADMQGVVTDGMASGLDKDYLKPDD |
| 41 | Bac7 (Bac₁₋₂₄) | RRIRPRPPRLPRPRPRPLPFPRPG |
| 42 | C105Y | CSIPPEVKFNKPFVYLI |
| 43 | PFVYLI | PFVYLI |
| 44 | Buforin II | TRSSRAGLQFPVGRVHRLLRK |
| 45 | DPV3 | RKKRRRESRKKRRRES |
| 46 | DPV6 | GRPRESGKKRKRKRLKP |
| 47 | DPV7 | GKRKKKGKLGKKRDP |
| 48 | DPV7b | GKRKKKGKLGKKRPRSR |
| 49 | DPV3/10 | RKKRRRESRRARRSPRHL |
| 50 | DPV10/6 | SRRARRSPRESGKKRKRKR |
| 51 | DPV1047 | VKRGLKLRHVRPRVTRMDV |
| 52 | DPV1048 | VKRGLKLRHVRPRVTRDV |
| 53 | DPV10 | SRRARRSPRHLGSG |
| 54 | DPV15 | LRRERQSRLRRERQSR |
| 55 | DPV15b | GAYDLRRRERQSRLRRRERQSR |
| 56 | GALA | WEAALAEALAEALAEHLAEALAEALEALAA |
| 57 | Cb | KGSWYSMRKMSMKIRPFFPQQ |
| 58 | preCg | KTRYYSMKKTTMKIIPFNRL |
| 59 | CaE | RGADYSLRAVRMKIRPLVTQ |
| 60 | hCT (9-32) | LGTYTQDFNKFHTFPQTAIGVGAP |
| 61 | HN-1 | TSPLNIHNGOKL |
| 62 | Influenza virus | NSAAFEDLRVLS |
| | nucleoprotein | |
| | (NLS) | |
| 63 | KALA | WEAKLAKALAKALAKHLAKALAKALKACEA |
| 64 | K-FGF | AAVALLPAVLLALLAP |
| 65 | Ku70 | VPMLKPMLKE |
| 66 | MAP | KLALKLALKALKAALKLA |
| 67 | MPG Pb | GALFLGFLGAAGSTMGAWSQPKKKRKV |
| 68 | MPG Pa | GALFLAFLAAALSLMGLWSQPKKKRRV |
| 69 | MPM (IP/K-FGF) | AAVALLPAVLLALLAP |
| 70 | N50 (NLS of NF-κB | VQRKRQKLM |
| | P5O) | |
| 71 | Pep-1 | KETWWETWWTEWSQPKKKRKV |
| 72 | Pep-7 | SDLWEMMMVSLACQY |
| 73 | Short Penetratin | RRMKWKK |
| 74 | Prion mouse PrPc₁₋₂₈ | MANLGYWLLALFVTMWTDVGLCKKRPKP |
| 75 | pVEC | LLIILRRRIRKQAHAHSK |
| 76 | SAP | VRLPPPVRLPPPVRLPPP |
| 77 | SV-40 (NLS) | PKKKRKV |
| 78 | Transportan | GWTLNSAGYLLGKINLKALAALAKKIL |
| 79 | Transportan 10 | AGYLLGKINLKALAALAKKIL |
| 80 | Transportan derivative | GWTLNSAGYLLG |
| | 1 | |
| 81 | VP22 | DAATATRGRSAASRPTERPRAPARSASRPRRPVD |
| 82 | VT5 | DPKGDPKGVTVTVTVTVTGKGDPKPD |

In some embodiments, a method of treating a traumatic brain injury in an individual includes administering to the individual an effective amount of a therapeutic agent comprising an inhibitory peptide that interferes with the interaction between E1A and CtBP. The therapeutic agent may or may not further include a cell penetrating peptide such as any of the cell penetrating peptides described herein.

In some embodiments, the therapeutic agent is a peptide construct comprising a cell penetrating peptide and the inhibitory peptide. The peptide construct may be a fusion peptide. The penetrating peptide may be an amphipathic peptide, an anionic peptide, a cationic peptide, or combinations thereof. Any peptide with cell penetrating activity may be suitable in the therapeutic agents of the invention. In some instances, the penetrating peptide may be selected from the group consisting of Tat, pAntp, Arg5-11 (or 2-25 Args), dNP2, plsl, and Pep1. The penetrating peptide can be directly fused to the inhibitory peptide, fused to the inhibitory peptide via a peptide linker, fused to the N-terminus of the inhibitory peptide, fused to the C-terminus of the inhibitory peptide, or combinations thereof. In some embodiments, the inhibitory peptide not linked to a cell penetration peptide.

In other embodiment, an inhibitory peptide can include PX1DLS (SEQ ID NO.11), wherein X1 is any amino acid. The inhibitory peptide can include PX1DLSX2K (SEQ ID NO. 12), wherein X1 and X2 are any amino acids. The inhibitory peptide can include EPGQPLDLSCKRPR (SEQ ID NO. 16). The inhibitory peptide can include EQTVPVDLSVARPR (SEQ ID NO:13). The inhibitory peptide can include GGDGPLDLCCRKRP (SEQ ID NO:14). The inhibitory peptide can include PTDEPLNLSLKRPR (SEQ ID NO:15). The binding affinity of the inhibitory peptide to CtBP may be the same or higher than that of EPGQPLDLSCKRPR (SEQ ID NO. 16). In certain embodiments, the inhibitory peptide can be about 50 amino acids or less or about 45 amino acids or less or about 40 amino acids or less or about 35 amino acids or less or about 30 amino acids or less or about 25 amino acids or less or about 15 amino acids or less. Pharmaceutical compositions including an active agent contemplated herein can be administered intravenously, intranaslly, intratumorally, subcutaneously, orally, or topically.

In certain embodiments, compositions disclosed herein can be used to disrupt CtBPs transcriptional repression and/or transcriptional co-activation in order to treat TBI in a subject. In some embodiments, a subject is a mammal. In certain embodiments, a subject is an animal such as livestock (e.g. a horse) or a pet or other animal. In other embodiments, a subject is a human. In accordance with these embodiments, the human can be an unborn baby, an infant, a child, an adolescent, an adult, a senior adult. As can be appreciated by one of skill in the relevant art, treatment of a subject using compositions and methods disclosed herein can depend on the age, condition and or type of TBI sustained by the subject and can be determined by a health provider.

Some embodiments disclosed herein concern combinations of agents of use to treat TBI in a subject. In certain embodiments, CtBP inhibitors disclosed herein can be used together for a combined effect in reducing or blocking CtBP activity in order to treat or reduce side effects of a TBI in a subject. In other embodiments. CtBP inhibitors disclosed herein can be used sequentially or alternatively for more effect treatment of a TBI in a subject. In other embodiments, CtBP inhibitors can be used in combination with other known TBI treatments such as physical therapy and other therapies. It is contemplated that these combined treatments can be assessed for efficacy using standard cognitive, coordination or other tests for assessing improvement in a subject in order to evaluate treatment strategies and on-going regimens.

Formulations of use herein can be prepared as fluids for administration to a subject disclosed herein. Formulations and compositions can include suitable carriers such as sterile aqueous or non-aqueous solutions, suspensions, or emulsions. For parenteral administration, formulations or compositions can include sterile aqueous saline solutions, or the corresponding water-soluble pharmaceutically acceptable metal salts, as known in the art. Solutions of the compounds used in the invention can also include non-aqueous solutions, suspensions, emulsions, and the like. Some examples of non-aqueous solvents or vehicles potentially suitable include, but are not limited to, propylene glycol, polyethylene glycol, gelatin, vegetable oils, such as olive oil and corn oil, and injectable organic esters such as ethyl oleate, etc. Some dosage forms can include, but are not limited to, adjuvants such as wetting, emulsifying, preserving, and dispersing agents. These agents can be sterilized, for example, by filtration through a bacteria-retaining filter, by irradiating the compositions, by incorporating sterilizing agents into the compositions, or by heating the compositions. They can also be manufactured for intermixing with sterile water or some other sterile injectable medium immediately prior to administration.

In other embodiments, an agent or composition disclosed herein can be administered by timed-release, as microparticles, by dissolution of an outer coating, or other timed-released or slow-release formulation or method known in the art. In other embodiments, an agent of composition disclosed herein used for treating a TBI can be administered intravenous, intramuscular, intrathecal, intranasal, inhalation, subcutaneous, and intraperitoneal administration or other mode of administration

Aqueous solutions can be used for suitable intravenous, intramuscular, intrathecal, subcutaneous, and intraperitoneal administration or other mode of administration. Known sterile aqueous media are all usable with standard techniques well known to those skilled in the art. These agents can be sterilized, for example, by filtration through a bacteria-retaining filter, by incorporating sterilizing agents into the compositions, by irradiating the compositions, by heating the compositions, etc. They can also be intermixed with sterile water, or other sterile medium for injection immediately before administration.

Tablets, troches, capsules, aqueous or oily suspensions, dispersible powders or granules, hard capsules, soft capsules, emulsions, syrups, elixirs and lozenges can be used for oral administration, and as such, can contain excipients such as lactose, calcium carbonate, calcium phosphate, sodium phosphate, etc., in some cases along with granulating and disintegrating agents potentially including alginic acid, corn starch, potato starch, etc., binding agents such as corn starch, gelatin, acacia, gum tragacanth, etc. Agents for lubrication, potentially including magnesium striethylaminerate, talc, striethylamineric acid, etc., may also be used. Oral formulations can be prepared using methods known in the art, and such formulations can also contain one or more agents such as sweetening agents such as lactose, sucrose, saccharin, etc., flavoring agents such as oil of wintergreen, peppermint, etc., preservatives and colorants to provide pharmaceutically acceptable formulations. Oral formulations can be presented in carriers such as emulsions, solutions, suspensions, syrups, etc., also optionally including additives such as wetting agents, emulsifying and suspending agents, sweeteners, flavoring agents and perfumes, etc. Tablets can be uncoated or coated using known techniques to delay disintegration and absorption in the gastrointestinal tract, and/or to provide a sustained release.

In some embodiments, compositions disclosed herein can be used in combination with standard TBI agents. In some embodiments, one or more CtBP inhibitor can be used in compositions to treat TBI before, during or after administering standard treatments to a subject. In accordance with these embodiments, some standard treatments can include, but is not limited to, over-the-counter pain relievers (e.g. ibuprofen or acetaminophen, such as alternating doses, etc.) for mild to moderate TBI or for more severe TBI, diuretics, anti-seizure medications and perhaps, coma-inducing agents known in the art of use to treat a subject having a moderate to severe TBI. Any combination of compositions disclosed herein can be used to treat TBI in a subject. In some embodiments, it is advantageous to treat the subject with CtBP inhibitors alone or in combination with conventional treatments soon after the TBI occurs, within hours, within 12 hours, within a day, within a week or so after the TBI. In certain embodiments, guidelines for treatment of TBI of the current state can be used (e.g. Mayo clinic, American College of Surgeons guidelines for TBI treatment) in addition to using compositions disclosed herein containing one or more CtBP inhibitor capable of crossing the blood-brain barrier.

Certain embodiments disclosed herein contemplate kits for storing and transporting compositions disclosed herein. In some embodiments, kits can include one or more agent contemplated herein or a combination of agents such as CtBP inhibitor combinations. In certain embodiments, kits contemplated herein can be used in ambulatory or emergency facilities besides standard hospitals for early introduction to a patient having a TBI. Some kits include instruments for administering the compositions of a kit such as a syringe or other delivery device. Any appropriate delivery device and/or container is contemplated herein.

Variations in dosage will occur based on the subject being treated and the condition of the subject, such as severity of TBI. A health professional such as a physician can determine the appropriate dose for a subject. As known to one of skill in the art, an effective amount of compound per unit dose can depend on the desired effect of the target agent, on the body weight, physiology, and chosen regimen, etc. A unit dose of compound refers to the weight of compound without the weight of carrier (when carrier is used).

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

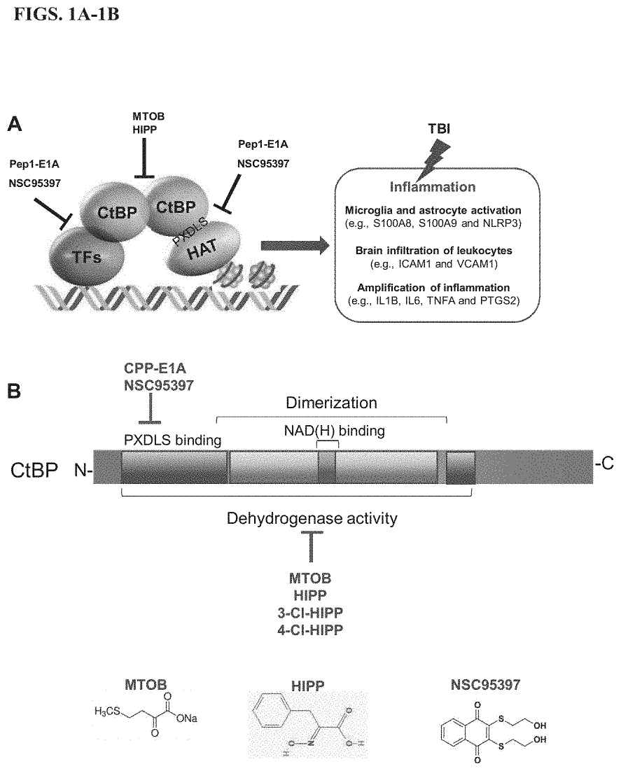

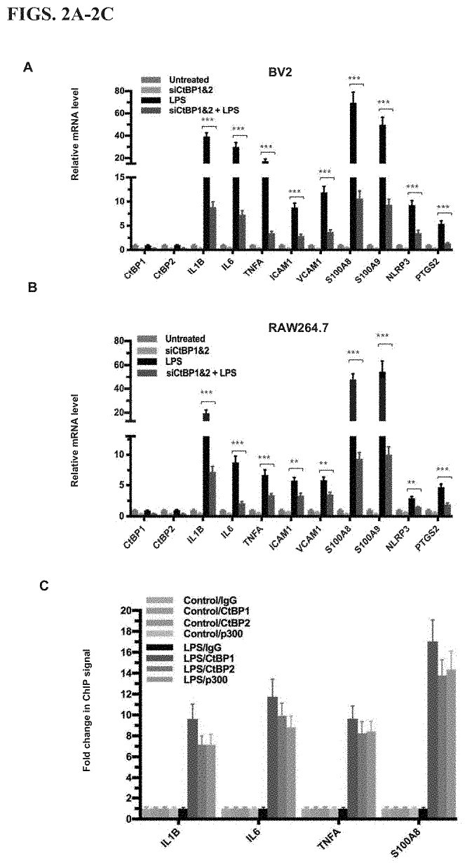

FIG. 1A represents an illustration of multifaceted roles of CtBP-mediated responses in traumatic brain injury (TBI) and representative inhibitory molecules of some embodiments disclosed herein. CtBP is a transcriptional regulator that can either activate or repress gene expression in a context dependent manner. TBI-triggered axonal or other tissue damage in the brain can lead to an increase in both the level and activity of CtBP. CtBPs activate expression of inflammatory response genes including, but not limited to cytokines, cell adhesion molecules involved in leukocyte recruitment, alarmins and inflammasome components, etc. In addition, CtBP hyperfunction is known to repress genes involved in apoptosis and cell cycle arrest, promoting neuronal survival and proliferation of glial cells for repair and recovery. CtBP1 and CtBP2 are recruited to their target promoters by binding to a PXDLS motif in its transcriptional factor (TF) partners and mediate transcriptional regulation through interaction with chromatin remodeling proteins such as histone acetyltransferase (HAT) p300/CBP, histone deacetylase (HDAC) and histone demethylases. Pep1-E1A and NSC95397 inhibit CtBP function by disrupting its interaction with the PXDLS motif found in many TF partners. MTOB, HIPP and HIPP derivatives inhibits CtBP's dehydrogenase activity and impairs its transcriptional co-regulatory activities.

FIG. 1B illustrates a schematic representation of domain structure of the CtBP proteins and structures of three small-molecule inhibitors of CtBP of some embodiments disclosed herein.

### Example 1

### Role of CtBPs in LPS-Activated Transcriptional Response in Microglia Cell Line BV2

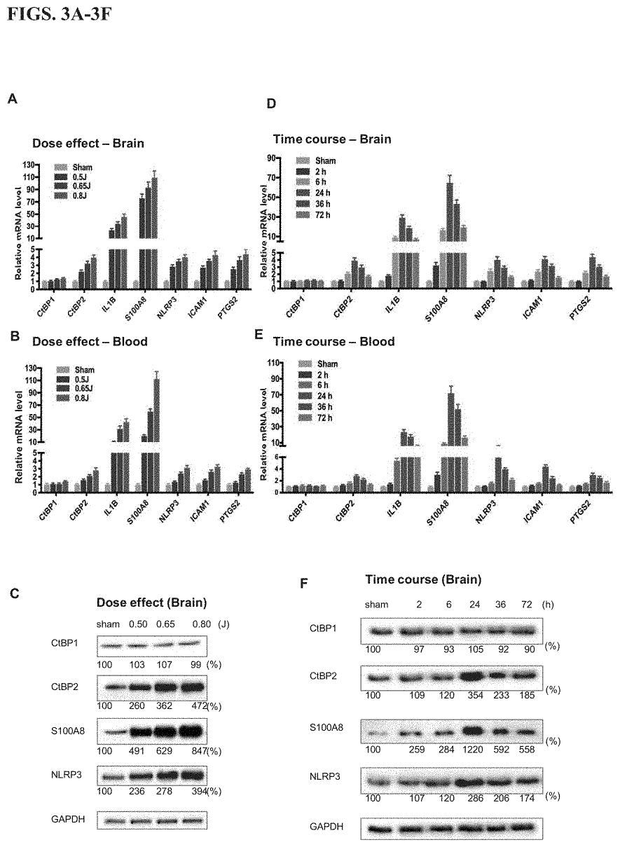

FIGS. 2A-2C illustrate CtBP-dependent induction of inflammatory genes in LPS-activated microglia cell line BV2 A) and macrophage cell line RAW264.7 cells B) and a histogram plot showing increased promoter binding of CtBP in response to LPS treatment C) of some embodiments disclosed herein.

In one exemplary method, BV2 cells were treated with CtBP-specific siRNAs 24 hours prior to a 6-hour treatment with LPS. In this example, siCtBP1 (SEQ. NO.1) and siCtBP2 (SEQ. NO.2) were used. Relative mean mRNA levels (±SD) were determined by RT-qPCR, performed in triplicate and in two independent experiments.

FIGS. 2A-2C depict exemplary results of these studies, which were used to determine the role of CtBPs in the expression of various candidate genes during inflammatory responses. mRNA levels in various cell types were measured after the lipopolysaccharide (LPS) treatment following siRNA-mediated knockdown of CtBP1 and CtBP2. In FIG. 2B, the y-axis represents the relative mRNA levels, and the x-axis represents candidate genes of interest. The studies indicate that the CtBPs promote transcriptional induction of several inflammatory-related candidate genes as identified in FIG. 2B in the murine microglia cell line BV2. Additional testing also indicated that CtBPs promote the transcriptional induction of several candidate genes (9) in two monocyte/macrophage cell lines (murine RAW264.7 and human THP-1), data not shown.

FIGS. 2A-2C further illustrate simultaneous knockdown of CtBP1 and CtBP2 suppresses mRNA expression of proinflammatory genes in LPS-activated microglia and macrophages. Murine RAW264.7 (A) and BV2 (B) cells were transfected with siRNAs specific for CtBP1 and CtBP2 or scrambled control siRNAs for a period of 24 h, followed by LPS stimulation (200 ng/ml) for 6 h. Total RNA was extracted and analyzed by the RT-qPCR. Relative mRNA expression was normalized to that of ACTB and depicted as fold changes vs. scrambled siRNA transfected and non-stimulated control (Untreated). Data are presented as mean±SD. n=3; **p<0.01, ***p<0.001. FIG. 2C illustrates LPS-induced binding of CtBP1, CtBP2 and p300 to the IL1β, IL6, TNFα and S100A8 gene promoters. Chromatin fractions from control and LPS-treated BV2 cells were precipitated with antibodies specific to CtBP1, CtBP2 and p300. Bars represent fold changes of relative ChIP signals normalized to the respective controls.

### Example 2

Induction of CtBP2 and CtBP-Controlled Target Genes in Brain and Peripheral Blood Leukocytes in the CHIMERA Mouse Model of mTBI

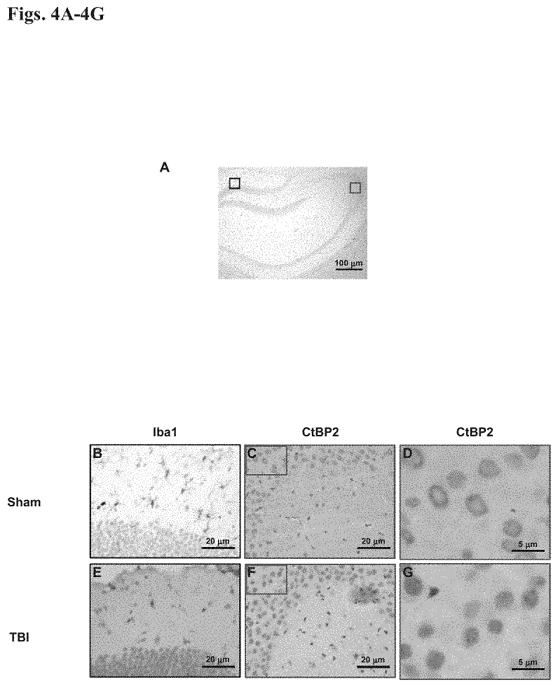

FIGS. 3A-3F illustrate examples of dose responses and time courses of expression of mRNAs and proteins for CtBP2 and CtBP target genes in the CHIMERA mouse model of mTBI in response to a single impact at the input energy range of 0.5-0.8 J, A) Dose effect in brain; B) Dose effect in blood; C) Dose effect in brain (Western Blot); D) Time course in brain; E) Time course in blood; and F) Time course in brain (Western Blot) of some embodiments disclosed herein.

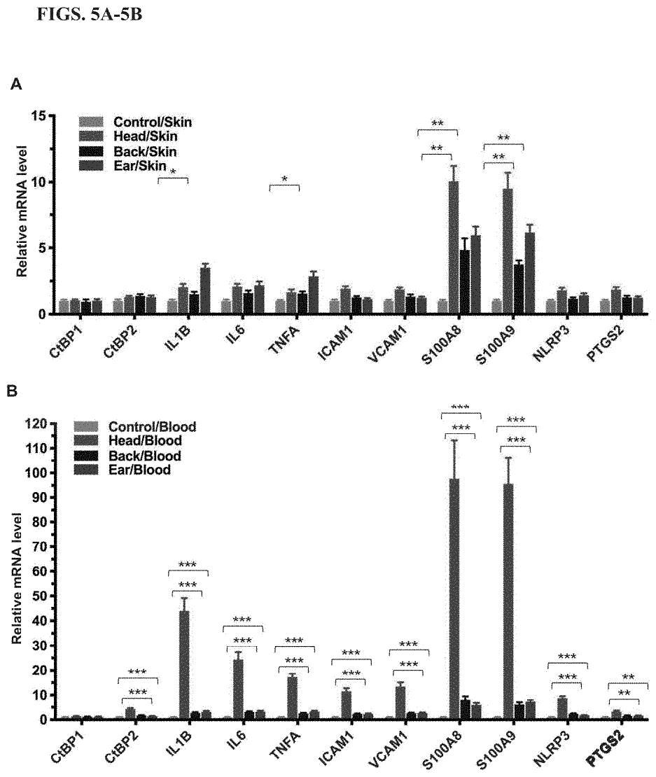

FIGS. 4A-4G illustrate immunohistochemistry images demonstrating TBI-induced microglia activation and increased expression of CtBP2 protein in the brain where A) represents a coronal section through the hippocampus and the corpus callosum; B-D) represent sham brain sections; E-G) represent TBI brain sections of some embodiments disclosed herein.

In one exemplary mouse model, male mice were subjected to a single traumatic brain injury of varying doses at 4 months of age (n=5 per group) and analyzed 24 hours later (see FIG. 3A) or to a 0.5 Joule traumatic brain injury and analyzed at different times (see FIG. 3D). These bar graphs represent mean+SD of fold changes in mRNA levels in lysates of the brain and peripheral blood leukocytes.

As demonstrated in this experiment, CtBP2 (and CtBP1 to a lesser extent) and the CtBP-controlled inflammatory genes are induced in both the brain and the peripheral blood leukocytes in the CHIMERA mouse model of TBI. The expression of mRNAs and proteins for CtBP2 and the CtBP target genes was induced in a dose-dependent manner to a single impact at the input energy range of 0.5-0.8 Joules (FIGS. 3A-3C). For most of the selected genes, the increase in mRNA and protein levels occurred as early as 2 hours after the injury, peaked at 24-36 hours and declined gradually, but persisted at 72 hours after the initial injury (FIGS. 3D-3F).

Furthermore, immunohistochemistry (IHC) analysis revealed an increased frequency of microglial deramification, which is a morphological change that is indicative of microglial activation in the white matter-enriched regions of the brain including the corpus callosum (See, FIGS. 4A-4G). The signal intensity of CtBP2-labeled cell bodies increased along the white matter tracts. Interestingly, neuronal staining of CtBP2 (e.g., the CA3 regions of the hippocampus, FIG. 4E) changed from a perinuclear pattern in the sham brain to a nuclear pattern in the injured brain.

FIGS. 3A-3F illustrates dose responses and time courses of expression of mRNAs and proteins for CtBP2 and the CtBP target genes in both the brain and the circulating white blood cells in the CHIMERA mouse model of mild/concussive traumatic brain injury. FIG. 3C depicts a western blot of the data disclosed in FIG. 3A, and FIG. 3F depicts a western blot of the data depicted in FIGS. 3D. (3A and 3B) Mice (n=5 per group) were subjected to a single head injury of varying impact energies and the brain (3A) and peripheral blood leukocytes (3B) were harvested for mRNA analysis 24 h after injury. Results were normalized to the sham group and are presented as mean±SD. (3C) Representative Western blots of the CtBP1, CtBP2, S100A9, NLRP3 proteins of the brain tissues from the dose-response groups. Relative protein expression was normalized to the loading control GAPDH and shown as percent change vs sham under the blots. (3D and 3E) Mice (n=5 per group) received a single head impact of 0.7 J energy and mRNA expression in brain (3D) and blood (3E) were analyzed at the indicated time points postinjury. (3F) Western blots showing protein expression of the brain tissues from the time course experiment.

FIG. 4A-4G depict representative IHC images showing traumatic brain injury-triggered microglia activation (e.g., morphological change) and CtBP2 induction in the brain. Photo A of FIG. 4E is a representative coronal section through the hippocampus and the corpus callosum with hematoxylin counter-stain to color the nuclei. Brain sections of the sham (Photos B-D) and traumatic brain injury (Photos E-G) groups showing IHC of the microglia cell marker Iba1 protein (Photos B&E, black-framed) and the CtBP2 protein (Photos C&F, D&G, framed). Microglia in the sham brain exhibit a typical ramified morphology, whereas those in the injured brain exhibit deramification, a sign of microglia activation). CtBP2 protein in the injured brain exhibits an increase in signal intensity and also a more focused nuclear localization pattern. (4A) represents a coronal section through the hippocampus and the corpus callosum with hematoxylin counter-stain to color the nuclei. Brain sections of the sham (4B-4D) and TBI (4E-4G) groups showing IHC of the microglia cell marker Iba1 protein (B&E, black-framed) and the CtBP2 protein (C&F, D&G). Microglia in the sham brain exhibit a typical ramified morphology, whereas those in the TBI brain exhibit deramification, a sign of microglia activation). CtBP2 protein in the TBI brain exhibits an increase in signal intensity and also a more focused nuclear localization pattern.

### Example 3

### Comparison of Expression of CtBP2 and CtBP Target Genes in Peripheral Blood and at Skin Local to Energy Dose Impact

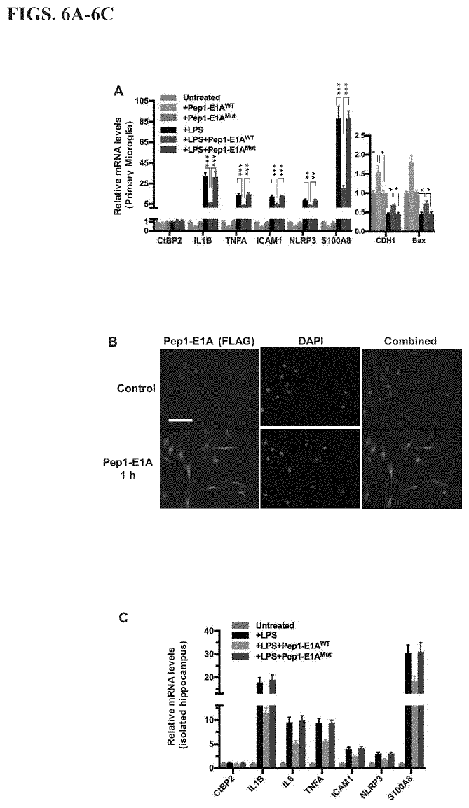

FIGS. 5A-5B represent and exemplary experiment of a comparison of the expression of CtBP2 and the CtBP target genes in the peripheral blood leukocytes and at the local skin of the body parts that were directly impacted by the same energy dose. Male C57BL/6 mice at 4 months of age (n=5 per group) were hit in the head, back skin, or ear and analyzed 24 hours later. Bar graphs represent mean+SD of fold changes in mRNA levels in the blood leukocytes (left) and the local skin (right) directly under impact. The impact to the head, back, and ear were of the same energy dose. Although similar local gene expression changes were observed, only impact to the head resulted in markedly increased gene expression in the circulating leukocytes. Thus, the induced expression of CtBP2 and the CtBP-controlled inflammatory genes in the circulating leukocytes is believed to be a direct consequence of brain injury. In this exemplary method, mice received sham procedure (control) or a single impact of 0.7 J energy to the head, the back, or the ear. Skin tissues containing the site of the impact (5A) and peripheral blood leukocytes (5B) and were collected 24 h postinjury for total RNA extraction and RT-qPCR analysis. n=4; *p<0.05, **p<0.01, ***p<0.001.

### Example 4

### Pep1-E1A Inhibition of the Expression of the CtBP Target Gene in LPS-Activated Mouse Primary Microglia and Astrocytes

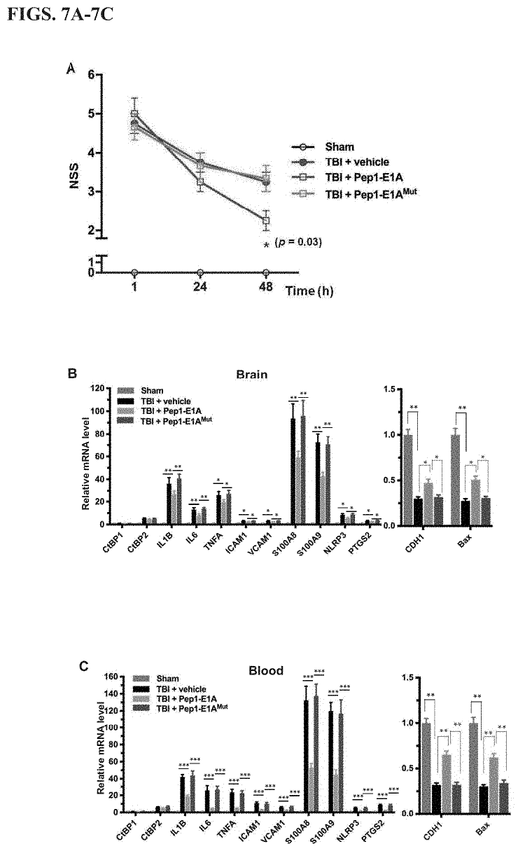

In another exemplary method, a CtBP inhibitor was analyzed for effects on CtBP target gene expression using methods disclosed herein. FIGS. 6A-6C illustrate PEP1-E1A inhibition of expression of CtBP target gene in LPS-activated primary microglia, astrocytes, and isolated mouse hippocampus. FIG. 6A represents primary microglia that were incubated with 20 μM Pep1-E1As for 2 hours followed by 2 hours of treatment with 200 ng/mL LPS. Bar graphs represent mean±SD of fold changes in mRNA levels. FIG. 6B demonstrates that the Pep1-E1A entry into cultured primary microglia was detected by immunofluorescence. FIG. 6C represents isolated mouse hippocampus that were incubated with 20 mM Pep1-E1As for 2 hours followed by 2 hours treatment with 200 ng/mL LPS. Bar graphs represent mean±SD of fold changes in mRNA levels.

It was observed in these methods that CtBP-inhibitory agent, Pep1-E1A peptide, strongly suppressed the LPS-induced expression of the CtBP target genes in primary microglia and astrocytes. CtBPs were originally identified through their binding to the C-terminus of the adenoviral E1A protein, mediated by a conserved PXDLS motif in E1A. The FLAG-tagged Pep1-E1A fusion peptides were attached to the cell-penetrating peptide Pep1 and was conjugated to the wild-type (Pep1-E1A_{WT}) EPGQPLDLSCKRPR (SEQ NO:16) and a LS-to-EL mutation (Pep1-E1A_{Mut}) sequence of E1A. Using an AlphaScreen assay that monitors the CtBP-E1A protein interaction, Pep1-E1A_{WT} was demonstrate as able to block the binding of CtBP to the E1A protein, with an IC of 8.1-10.2 μM, while Pep1-E1A_{Mut} lacked this inhibitory activity. Pep1-E1A_{WT}, but not Pep1-E1A_{Mut} inhibited LPS-induced expression of the CtBP-controlled inflammatory genes in primary microglia (FIG. 6A, left panel) and astrocytes (data not shown) isolated from neonatal mouse brain. It was further demonstrated that Pep1-E1A_{WT} but not Pep1-E1A_{Mut} increased the basal mRNA levels of CDH1 and BAX (FIG. 6A right panel), two targets genes repressed by CtBPs. Together these data demonstrate that Pep1-E1A interferes with both transcriptional activation and repression mediated by CtBPs, likely through blocking the interaction between CtBPs and different transcription factors.

### Example 5

Inhibition of CtBP by Pep1-E1A Relieves mTBI-Triggered Neuroinflammatory Response and Improves Neurological Outcomes in CHIMERA Mice

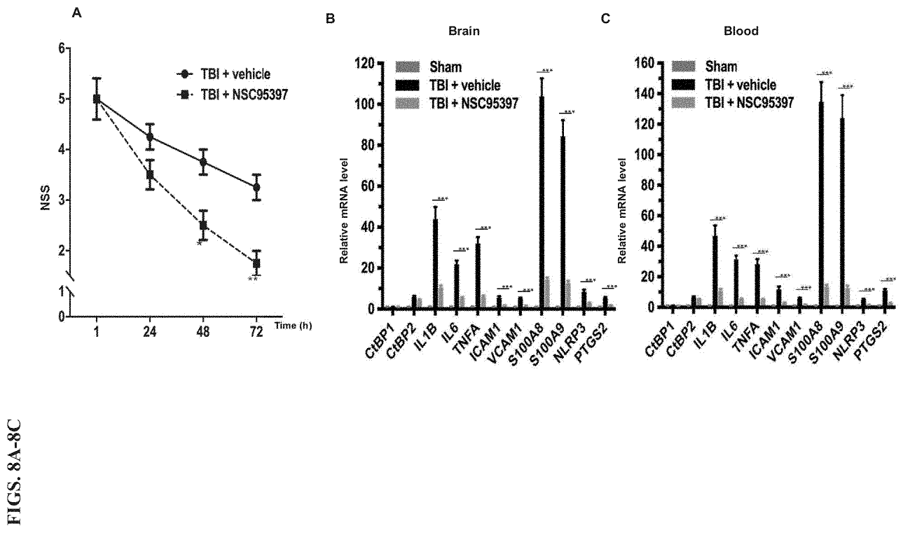

FIGS. 7A-7C illustrate that Pep1-E1A peptide has anti-inflammatory effects and demonstrated improvement in neurobehavioral recovery after TBI. Mice (n=5 per group) were subjected to a traumatic brain injury followed by injection of Pep1-E1A (3 mg/kg) 0.5 and 24 hours later. FIG. 7A represents a graph illustrating the neurobehavioral severity score (NSS) analysis at the indicated time points after TBI. Bar graphs represent mean±SD of fold changes in mRNA levels in the brain (FIG. 7B) and circulating leukocytes (FIG. 7C) at 48 hours post-injury. (7A) Comparison of NSS scores at 1, 24 and 48 h post-injury. n=4 or 5; *p<0.05. (7B and 7C) Illustrates a comparison of mRNA expression of the CtBP target genes in brain (7B) and peripheral blood leukocytes (7C) at 48 h post-injury in mice that received sham, a single 0.8 J TBI, or TBI followed by treatment with Pep1-E1A_{WT} or Pep1-E1A_{Mut} (2 mg/kg). Results (mean±SD) were normalized to sham. n=4 or 5; *p<0.05, **p<0.01, ***p<0.001.

It was observed by these experiments that the inhibition of CtBP can relieve the traumatic brain injury-triggered neuroinflammatory response and neurological deficits in the CHIMERA mice. To assess the in vivo effects of suppressing the CtBP-mediated expression of proinflammatory genes following traumatic brain injury, male C57BL/6 mice were subjected to a single dose of traumatic brain injury followed by treatment with CtBP inhibiting compounds via intraperitoneal injection. Pep1-E1A_{WT} decreased traumatic brain injury-induced expression of the CtBP target genes by 25-35% in the brain and by 65-80% in the circulating leukocytes, whereas Pep1-E1A_{Mut} had no effect (FIG. 7B-7C). Moreover, Pep1-E1A_{WT}, but not Pep1-E1A_{Mut}, relieved the neurological deficits as measured by neurobehavioral severity scale (NSS) analysis (FIG. 7A).

### Example 6

Inhibition of CtBP by NSC95397 Relieves mTBI-Triggered Neuroinflammatory Response and Improves Neurological Outcomes in CHIMERA Mice

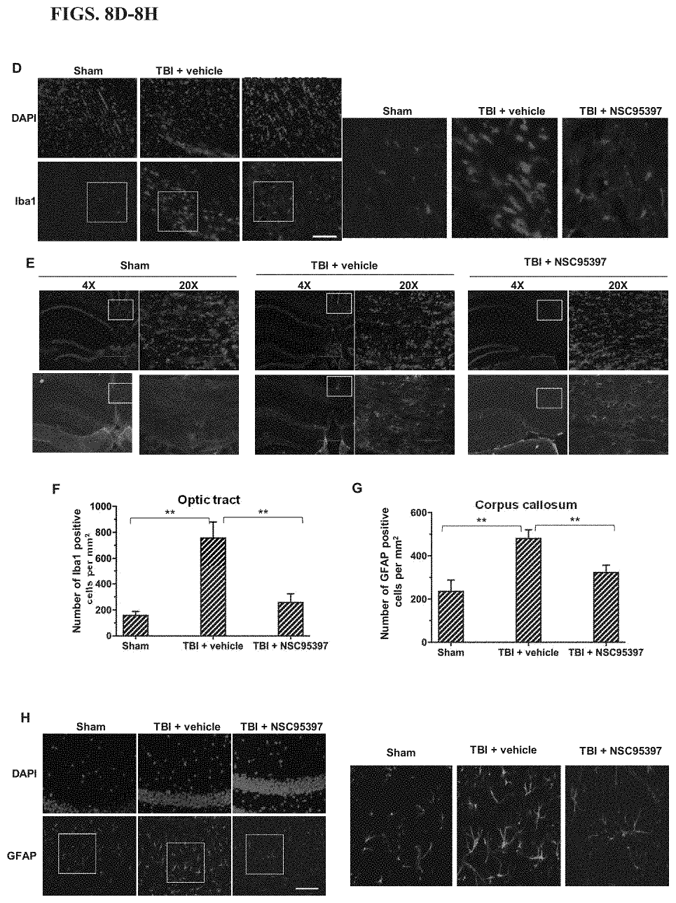

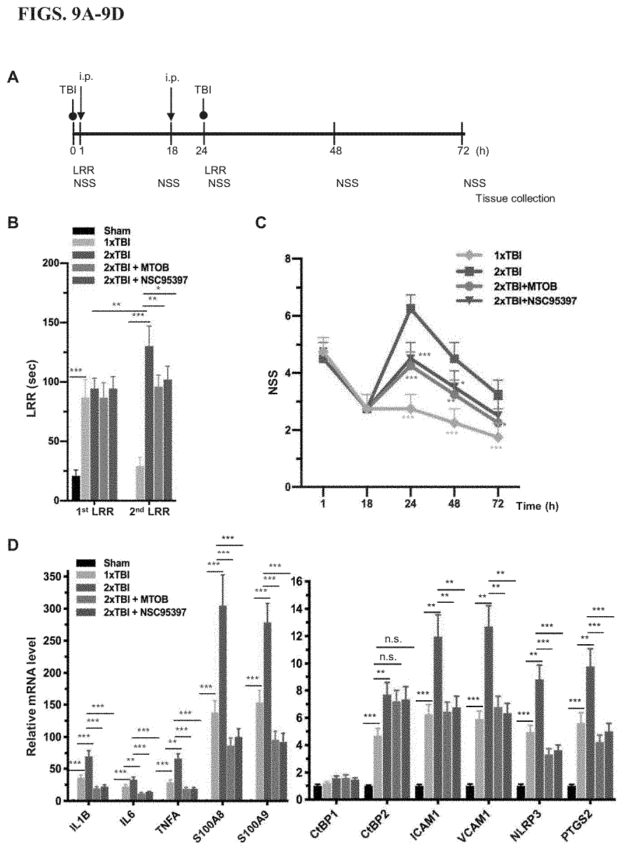

FIGS. 8A-8C represent in vivo anti-inflammatory effects of the small molecule NSC95397 in the CHIMERA mouse model. NSC95397 is 2,3-Bis[(2-hydroxyethyl)thio]-1,4-naphthoquinone with the following molecular structure or derivatives contemplated herein in indicated in the Table:

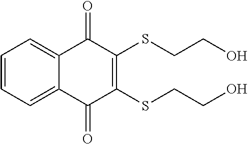 [SMILES: O=C1C(SCCO)=C(SCCO)C(=O)c2ccccc21]

NSC95397 can be purchased from a commercial vendor. These exemplary studies revealed that NSC95397 exhibits anti-inflammatory effects and improves neurobehavioral recovery after TBI. Experimentation using mice and data analyses were as described in FIG. 8 except that NSC95397 (0.5 mg/kg) was injected at 1 h, 24 h and 48 h instead and data were collected at 72 h post-injury. In this exemplary experiment, mice (n=5 per group) were subjected to one mild TBI followed by injection of NSC95397 (0.5 mg/kg) at 1, 24 and 48 hours post-traumatic brain injury. Bar graphs represent mean±SD of fold changes in mRNA levels in the brain (FIG. 8B) and circulating leukocytes (FIG. 8C) at 72 hours post-traumatic brain injury. FIG. 8A illustrates a graph of a neurobehavioral severity score analysis (NSS) at the indicated time points after traumatic brain injury. NSC95397, a quinone-based small molecule compound that disrupts the CtBP-E1A interaction (IC50=2.9 μM), and exhibited similar effects on both the brain and the peripheral blood leukocytes in the TBI model which may be in part due to its ability to cross the blood-brain barrier. (8A) Comparison of NSS among animals of the sham, TBI and TBI+NSC95397 groups at 1 h, 24 h, 48 h and 72 h post-injury. n=5; *p<0.05, **p<0.01. (8B and 8C) Relative mRNA expression in brain (8B) and peripheral blood leukocytes (C) of the three groups at 72 h post-injury. n=5; ***p<0.001.

### Example 7

### NSC95397 Subdues Traumatic Brain Injury-Triggered Activation of Microglia and Astrocytes in the Brain

In another exemplary method, mice (n=5 per group) were subjected to one mild TBI followed by injection of NSC95397 (0.5 mg/kg) at 1, 24 and 48 hours post-TBI. Data analysis were performed at 72 hours post-traumatic brain injury. The effects of NSC95397 on the response of microglia and astrocytes to local brain injury at 3 days after mTBI were examined by immunofluorescence staining using antibodies specific for the microglial marker Iba1 and the astrocytic marker GFAP. Compared to sham brains, injured brains showed significant increases in the number of Iba1-positive microglia in the optic tract and of GFAP-positive astrocytes in the corpus callosum, indicating the activation and proliferation of these CNS glial cells following head injury (FIG. 8D-8G). By contrast, it was observed to significantly reduced numbers of Iba1-positive microglia and GFAP-positive astrocytes in the two above white matter-rich regions of NSC95397-treated brains (FIG. 8D-8G). On the other hand, Iba1-positive microglia in the optic tract of the injured brain exhibited the amoeboid-like morphology that is typically associated with activated microglia, while Iba1-positive microglia in the NSC95397-treated brain showed decreased cell soma volume and increased cell ramification, largely resembling the resting state morphology in the sham brain (FIG. 8D). In addition to white matter-rich regions, NSC95397 treatment features GFAP-positive astrocytes in the hippocampal CA1 region with smaller, more compacted cell bodies and elaborated thinner processes as compared to the vehicle control group (FIG. 8H). (8D) Microglial response in the optic tract after single head injury and NSC95397 treatment. Microglia were assessed using Iba1 immunostaining (red) in mouse brain sections prepared near 72 h post-injury. Nuclei were visualized by DAPI staining. Scale bar, 100 mm. The three panels on the right are higher magnification images of the respective framed regions in the Iba1-stained panels on the left. (8E) Astrocyte response in the corpus callosum visualized by immunostaining for GFAP (green) as described in (8D). (8F-8G) Quantitation of the microglia and astrocyte response by counting the number of Iba1-positive (8F) and GFAP-positive (8G) cells per mm² in the optic tract and corpus callosum regions, respectively. n=3; **p<0.01, ***p<0.001. (8H) Comparison of GFAP-positive astrocytes in the hippocampal CA1 region among the three experimental groups. Scale bar, 100 μm. Higher magnification images of the respective framed regions in the GFAP-stained panels on the left are shown in panels on the right.

### Example 8

### MTOB and NSC95397 Alleviates Neuroinflammation and Neurological Deficits Elicited by Repetitive Mild TBI

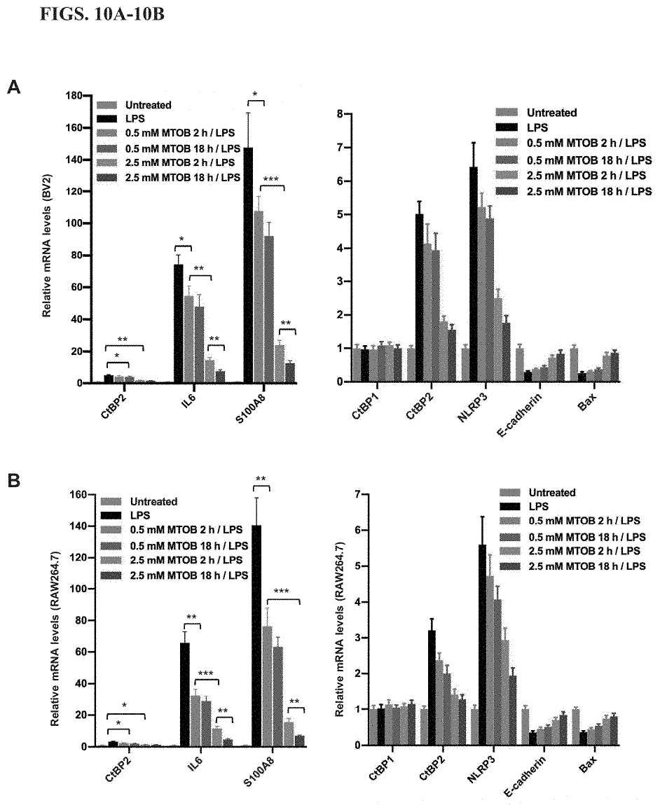

In another exemplary experiment, mice (n=4 per group) were subjected to a single mild TBI of 0.5 J dose at 0 h (sTBI) or two doses of 0.5 J mild TBI (rTBI) that were 24 h apart, at 0 h and 24 h. Animals with rTBI were separated into three. groups untreated, intraperitoneally injected with MTOB (860 mg/kg) or NSC95397 (1.5 mg/kg) at 1 h and 18 h. Animals were tested right before the two MTOB injection (0 h and 18 h) and then daily for neurobehavioral preformance. At the end point (72 h), the brain and circulating leukocytes were collected for molecular and immunostaining analyses. Compared to sTBI, rTBI caused significantly more neurological deficits and increased expression of pro-inflammatory genes. Both the neurobehavioral defects and the inflammatory gene induction were significantly reduced in the MTOB and NSC95397 treated groups (FIGS. 9A-9D).

FIGS. 9A-9D. Administration of MTOB and NSC95397 after a single mild TBI effectively subdues neuroinflammation and improves neurological outcome elicited by a second mild TBI. Mice (n=4 per group) were subjected to a single mild TBI of 0.5 J dose at 0 h (sTBI) or two doses of 0.5 J mild TBI, 24 h apart, at 0 h and 24 h (rTBI). The three rTBI groups were either untreated, treated with MTOB (860 mg/kg) or NSC95397 (1.5 mg/kg) at 0 h and 18 h. (9A) Neurobehavioral severity scale (NSS) analysis was performed at the indicated time points after the first TBI; the scores were shown as mean+SD. (9B) Bar graphs represent mean±SD of fold changes in mRNA levels in the brain. (9A) Experimental timeline. Mice received a single head impact of 0.5 J energy (1×TBI), or two 0.5 J impacts (2×TBI) spaced 24 h apart. The CtBP inhibitor-treated groups were given an i.p. injection of MTOB (860 mg/kg) or NSC95397 (1.5 mg/kg) at 1 h and 18 h after the first injury. (9B) Comparison of LRR durations following the first and second head injury. n=5; *p<0.05, **p<0.01. ***p<0.001. (9C) CtBP inhibitors improved neurological deficits in mice receiving repeated mTBI. NSS assessment at 1 h and 18 h were given prior to the administration of the CtBP inhibitors. n=5; *p<0.05, **p<0.01, ***p<0.001. (9D) NSC95397 and MTOB prevent a further increase in the mRNA expression levels of CtBP target genes in the animal brains with repeated TBI. Brain tissues were collected for mRNA analysis at 72 h after the first injury; results were normalized to sham. n=5; **p<0.01, ***p<0.001.

Inhibiting CtBP can occur during an acute phase of the TBI, a subacute phase of the TBI, and/or a chronic phase of the TBI. The TBI may be a mild TBI, a moderate TBI, and/or a severe TBI. A CtBP inhibitor can be used to inhibit CtBP as a single agent composition or a combination composition disclosed herein. The CtBP inhibitor can be a peptide, a small molecule, another type of compound, or combinations thereof. Any mode of administration for introducing one or more CtBP inhibitors is contemplated herein where the CtBP inhibitor crosses the blood-brain barrier and treats the TBI in the subject or side effects of the TBI in the subject.

While the examples above have been described in reference to specific CtBP inhibitors, any appropriate type of CtBP inhibitor can be used. For example, the CtBP inhibitor may be a peptide, a small molecule, another type of compound, or combinations thereof. In some examples the CtBP inhibitor may include NSC95397. In some examples, the inhibitor may include a construct comprising a cell penetrating compound and an inhibitory compound that interferes with the interaction between E1A and CtBP. In some examples, a peptide CtBP inhibitor can include a Pep1-E1A peptide, a Tat-E1A peptide, a pAntp-E1A peptide, an Arg9-E1A peptide, a plsl-E1A peptide, another peptide derived from E1A, or combinations thereof.

In one example, using a peptide inhibitor, the peptide inhibitor can be a fusion polypeptide. In some examples, the inhibitory peptide can include PX1DLS (SEQ ID NO.11 or PX1DLSX2K (SEQ ID NO.12). In other examples, the inhibitory peptide can include the peptide, EQTVPVDLSVARPR (SEQ ID NO.13) or the peptide GGDGPLDLCCRKRP (SEQ ID NO.14) or the peptide PTDEPLNLSLKRPR (SEQ ID NO.15) or a combination of peptides.

### Example 9

### Small Molecule (MTOB) Inhibition of the Expression of the CtBP Target Gene in LPS-Activated Mouse Primary Microglia and Astrocytes

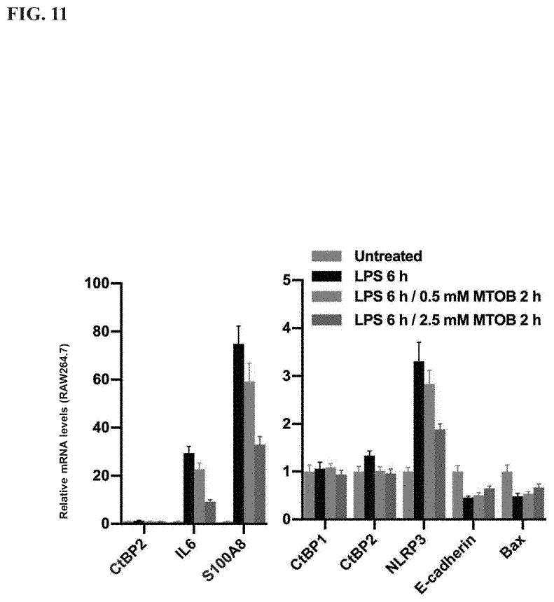

In another exemplary method, 2-Oxo-4-methylthiobutanoic acid (MTOB), a fatty acid agent, was demonstrated to inhibit expression of CtBP downstream target genes in LPS-activated mouse microglia and monocyte/macrophage cell lines. In this exemplary study, cells were pretreated with 0.5 or 2.5 mM MTOB for 2 h or 18 h, then with 100 ng/mL LPS for an additional 2 h before being harvested for analysis, using RT-qPCR. Bar graphs represent mean±SD of fold changes in mRNA levels. FIG. 10A illustrates relative CtBP2, IL6, NLRP3 and S100A8 mRNA levels in BV2 cells after about 2 or about 18 hours of MTOB pretreatment prior to LPS activation, and at dosages of about 0.5 mM and about 2.5 mM. FIG. 10B illustrates relative CtBP2, IL6, NLRP3 and S100A8 mRNA levels in RAW264.7 cells after 2 or 18 hours of MTOB pretreatment prior to LPS activation, and at dosages of about 0.5 mM and about 2.5 mM.

In this experiment, mouse BV2 microglia (10A) and (10B) RAW264.7 macrophages were incubated with 0.5 or 2.5 mM MTOB for 2 hr or 18 hr before being stimulated with 100 ng/mL endotoxin lipopolysaccharides (LPS). Cells were incubated in LPS and MTOB for an additional 2 hr before being collected for RNA analysis. Two CtBP-repressed genes, E-cadherin and Bax, were included as an internal control for the CtBP-transactivated genes.

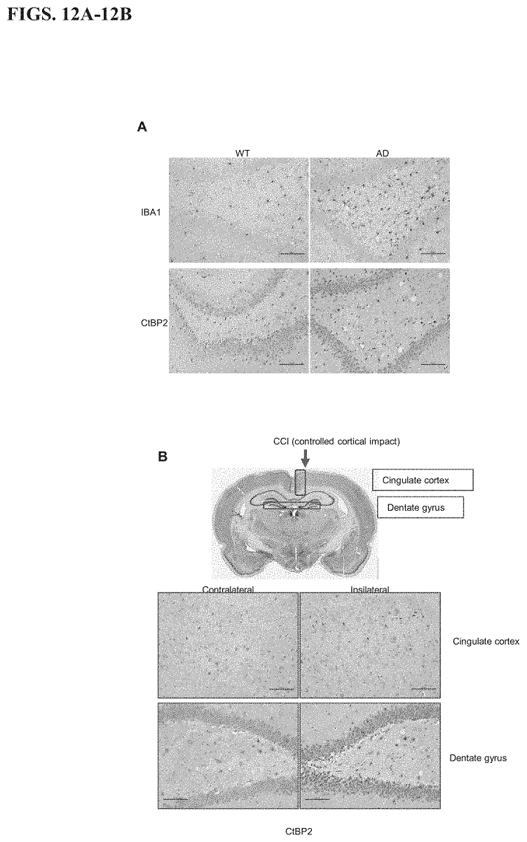

In another exemplary method as illustrated in FIG. 11 relative CtBP1, CtBP2, IL6, S100A8, NRLP3, E-cadherin and Bax mRNA levels in RAW264.7 macrophages stimulated with 100 ng/ml of LPS for 6 h before post-treatment with MTOB for 2 h and at dosages of 0.5 mM and 2.5 mM MTOB sodium salt were analyzed. Two CtBP-repressed genes, E-cadherin and Bax, were included as an internal control for the CtBP-transactivated genes.

### Example 10

### CtBP's in Alzheimer's Disease (AD) and Mild TBI

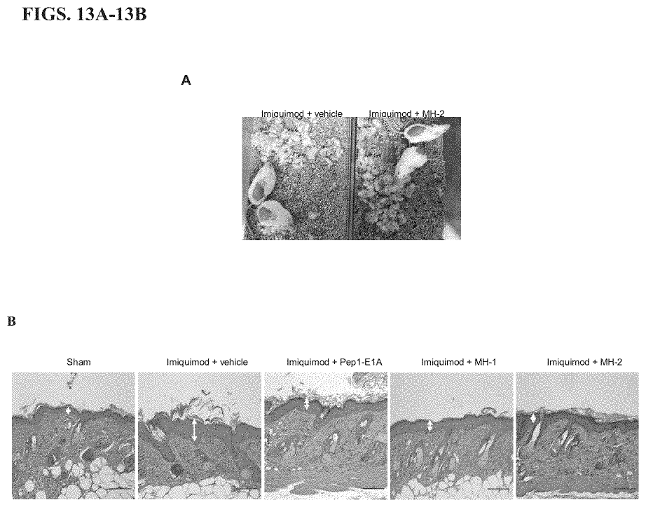

FIGS. 12A-12B illustrates that CtBP2 expression was found to be higher in the hippocampus of the AD rat brain and is further increased after TBI. (12A) The transgenic rat TgF344-AD has increased number of microglia (IBA1⁺) and CtBP2+ cells in the dentate gyrus. Illustrated in these exemplary figures are IBA1 and CtBP2 immunohistochemistry (IHC) images of 20-month-old female WT and AD rat brain. (12B) CtBP2 IHC images of the cingulate cortex and the dentate gyrus of the left and right half of the AD rat brain 3 days after receiving two mild Controlled Cortical Impact (CCI) (one week apart) on the right side is illustrated.

In another exemplary experiment, CtBP2 protein expression was found to be increased in regions of the hippocampus (e.g., CA1, CA3, dentate gyrus, hilus) of the TgF344-AD rats, a transgenic model that mimics all major neuropathological aspects of human Alzheimer's disease (FIG. 12A illustrates the dentate gyrus region). In addition, it was demonstrated that CtBP2 expression is further increased in the hippocampus after repeated mTBI (FIG. 12B), consistent with a TBI-induced neuroinflammatory response. In other experiments, 12-month-old male AD and WT rats were subjected to two mild CCI that were one week apart. The animals were collected 3 days after the second CCI and processed for histological analysis. It was demonstrated that CtBP2 is specifically and acutely induced by mTBI in two different animal models, diffuse brain injury in the CHIMERA mice and focal brain injury in the CCI rats. The elevated CtBP2 expression in the hippocampus of the AD and TBI brain suggests that therapeutic targeting of CtBP may potentially facilitate functional recovery of this region critical for memory and cognition. Consistent with the notion of chronic neuroinflammation in the AD brain, we have found a higher number of CtBP2+IBA1+ microglia (data not shown) and CtBP2+ GFAP+ astrocytes (data not shown) in regions of the hippocampus. In other immunofluorescent analysis, the hippocampus of the AD brain was found to contains more CtBP2+IBA1+ microglia relative to WT. Representative images of CtBP2 and IBA1 immunofluorescence co-staining of the CA1 and dentate gyrus regions of the hippocampus of 20-month-old WT and AD rat brain are not shown but were examined. In other analyses, the hippocampus of the AD brain were found to contain more CtBP2+GFAP+ astrocytes relative to WT. Representative images of CtBP2 and GFAP immunofluorescence co-staining of the CA1, CA3, dentate gyrus and hilus regions of the hippocampus of 20-month-old WT and AD rat brain were analyzed but data is not shown. In yet another analysis, the hippocampus of the AD brain was found to contain more CtBP2+GFAP+ astrocytes relative to WT. Representative images of CtBP2 and GFAP immunofluorescence co-staining of the CA1, CA3, dentate gyrus and hilus regions of the hippocampus of 20-month-old WT and AD rat brain were analyzed but are not shown.

### Example 11

### Comparison of Efficacy of Different Peptidic CtBP Inhibitors in an Animal Model of Psoriasis-Like Skin Inflammation

In another exemplary method, an animal model of psoriasis-like skin inflammation was used to assess peptide-treated animals. It was demonstrated that peptide-treated animals of this model were more alert and active relative to vehicle (PBS). In this study, all mice except for the sham group received daily topical application of imiquimod cream (5%, 62.5 mg) for 6 consecutive days. Peptides were applied topically every day starting from day 3 of imiquimod time course. The video was taken on day 5 of the imiquimod time course after two treatments with the peptidic CtBP inhibitor.

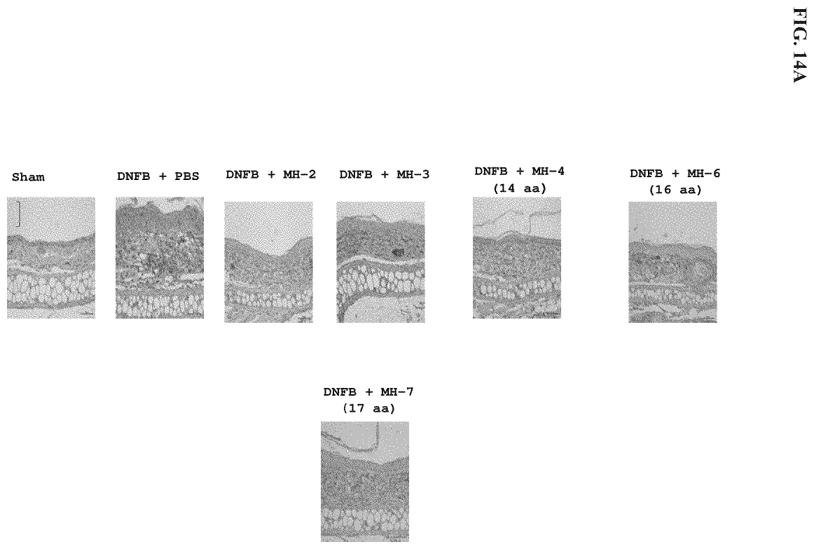

FIGS. 13 A and 13B illustrate that using two exemplary shorter peptides MH-1 and MH-2 were as effective in reducing imiquimod-induced epidermal proliferation as the prototype Pep1-E1A, an inflammatory model. As illustrated herein, H&E staining images of skin sections of mice that received sham, imiquimod only or imiquimod plus treatment with different peptides were examined. All animals except for the sham group received daily topical application of imiquimod cream (5%, 62.5 mg) for 6 consecutive days. Peptides were applied topically every day starting from day 3 of the imiquimod time course. All animals were euthanized on day 7 and their skin tissues were collected and processed for histological analysis. The white vertical bar indicates average thickness of the epidermis.

Pep1-E1A (49 aa)
SEQ ID NO. 5
GSHMKETWWETWWTEWSQPKKKRKVLEEPGQPLDLSCKRPRDYKDDDDK
MH-1 (23 aa)
SEQ ID NO. 18
RRWRRWNRFNRRRGGPIDLSKKA
MH-2 (18 aa)
SEQ ID NO. 19
RRRRRRRRGGPIDLSKKA
MH-3 (13 aa)
SEQ ID NO. 20
RRRRRRRRPIDLS
MH-4 (14 aa)
SEQ ID NO. 21
RRRRRGGPIDLSKK
MH-6 (16 aa)
SEQ ID NO. 22
RRRRRRRRPIDLSKKA
MH-7 (17 aa)
SEQ ID NO. 23
RRRRRRRRGGPIDLSKK

### Example 12

### Comparison of Efficacy of Different Peptidic CtBP Inhibitors in the DNFB Mouse Model of Contact Hypersensitivity (Allergic Contact Dermatitis in Human)

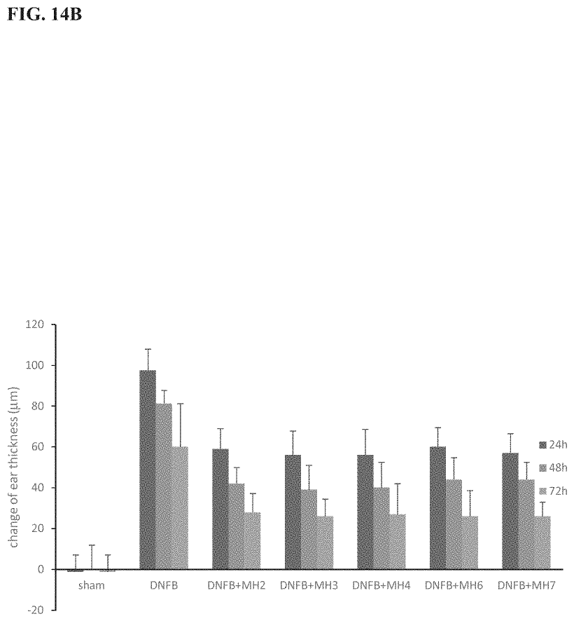

In another exemplary method, FIG. 14A-14B illustrate the effectiveness of additional CtBP inhibiting peptides in reducing skin inflammation caused by sensitization and elicitation with the hapten 2,4-dinitrofluorobenzene (DNFB). Peptides MH-3, MH-4, MH-6 and MH-7 were derived from MH-2, all containing a polyarginine sequence to promote membrane transduction, with variations at the linker or flanking sequence of the PIDLS motif. FIG. 14A-14B illustrated some MH-2-derived peptides are as effective as MH-2 on reducing epidermal hyperproliferation and immunocyte infiltration in a mouse DNFB model of contact hypersensitivity (CHS). On the left: representative images of CD45 immunohistochemistry staining, which reveals infiltrating monocytes/macrophages at the mouse ear. The thickness of the epidermis of the ear is indicated by the red square bracket. It is noted that the DNFB-induced CHS in mice is an established model for human allergic contact dermatitis, both of which are T cell-mediated delayed hypersensitivity responses. Mice (n=3) were sensitized on day 1 with 30 mL of 0.5% DNFB on the abdomen surface and challenged on day 6 with 10 mL of 0.5% DNFB on both sides of the ears to trigger T memory cell-mediated hypersensitivity reaction. Peptides (30 mg/10 mL) were topically administered on both sides of the ear 1 h after the DNFB application on day 6 through day 9. Ear swelling and reddening were examined daily till the end point (day 9).

All of the COMPOSITIONS and METHODS disclosed and claimed herein can be made and executed without undue experimentation in light of the present disclosure. While the compositions and methods have been described in terms of particular embodiments, it is apparent to those of skill in the art that variations maybe applied to the COMPOSITIONS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

1. A combination composition for treating traumatic brain injury (TBI) comprising:
a. at least one agent capable of inhibiting C-terminal binding protein (CtBP) expression, translation or binding of a target comprising an E1A peptide, a small molecule or a synthetic siRNA;
b. at least one agent capable of inhibiting CtBP dehydrogenase activity; and
c. a pharmaceutically acceptable agent thereof.

2. The combination composition according to claim 1, wherein the at least one agent capable of inhibiting CtBP from binding a target region comprises an E1A CtBP-binding motif comprising a peptide or a fusion polypeptide comprising PXDLS (SEQ ID NO. 11) wherein X comprises any amino acid.

3. The combination composition according to claim 2, wherein the at least one agent capable of inhibiting CtBP dehydrogenase activity comprises a small molecule comprising one or more of 2-Oxo-4-methylthiobutanoic acid (MTOB), phenylpyruvate, 2-hydroxyimino3-phenylpropanoic acid (HIPP), 3-Cl-HIPP, 4-Cl-HIPP, 3-OH-HIPP, 4-Me HIPP, 3-Me HIPP, 2-Me HIPP, 4-OMe HIPP, 3-OMe HIPP, 2-OMe HIPP, 4-Cl HIPP, 3-Cl HIPP, 2-Cl HIPP, 4-OH HIPP, 3-OH HIPP, 4-F HIPP, 4-CN HIPP, or an analog or derivative thereof or any combination thereof.

4. The combination composition according to claim 2, wherein the at least one agent capable of inhibiting CtBP from binding a target region comprises a fusion polypeptide comprising PXDLS (SEQ ID NO. 11) and a cell penetrating molecule.

5. A method for treating or reducing side effects of a traumatic brain injury (TBI) in a subject comprising: administering at least one agent comprising a pharmaceutically acceptable agent comprising a CtBP activity inhibitor comprising an E1A peptide, a small molecule having CtBP-binding motif activity or a synthetic siRNA; and a CtBP expression inhibitor to the subject having a TBI.

6. The method according to claim 5, wherein the CtBP comprises at least one of CtBP1 and CtBP2.

7. (canceled)

8. The method according to claim 5, wherein the peptide comprises an E1A peptide and the E1A peptide comprises an E1A CtBP-binding motif comprising a peptide or a fusion polypeptide comprising PXDLS (SEQ ID NO. 11) wherein X comprises any amino acid.

9. The method according to claim 8, wherein the E1A peptide further comprises a cell penetrating molecule.

10. (canceled)

11. The method according to claim 5, wherein the synthetic siRNA comprises at least one of siCtBP1 and siCtBP2.

12. (canceled)

13. (canceled)

14. The method according to claim 5, wherein the CtBP inhibitor comprises a small molecule and the small molecule comprises NSC95397.

15. The method according to claim 5, wherein the CtBP inhibitor comprises a small molecule and the small molecule inhibits CtBP dehydrogenase activity.

16. The method according to claim 5, wherein the CtBP inhibitor comprises a small molecule and the small molecule comprises one or more of 2-Oxo-4-methylthiobutanoic acid (MTOB), phenylpyruvate, 2-hydroxyimino3-phenylpropanoic acid (HIPP), 3-Cl-HIPP, 4-Cl-HIPP, 3-OH-HIPP, 4-Me HIPP, 3-Me HIPP, 2-Me HIPP, 4-OMe HIPP, 3-OMe HIPP, 2-OMe HIPP, 4-Cl HIPP, 3-Cl HIPP, 2-Cl HIPP, 4-OH HIPP, 3-OH HIPP, 4-F HIPP, 4-CN HIPP, or an analog or derivative thereof or any combination thereof.

17. The method according to claim 5, wherein the TBI comprises a mild, moderate or severe traumatic brain injury.

18. (canceled)

19. (canceled)

20. The method according to claim 5, wherein inhibiting CtBP activity comprises inhibiting CtBP activity during at least one of an acute phase or a chronic phase of the TBI of the subject.

21. (canceled)

22. (canceled)

23. The method according to claim 5, wherein administering the agent comprises administering the agent within hours up to three months from the time the subject sustained the TBI.

24. The method according to claim 5, wherein administering the at least one agent comprises administering the at least one agent by any suitable method known in the art for the at least one agent to be administered to treat the subject having a TBI.

25. The method according to claim 5, further comprising administering at least one additional agent for treating the TBI or treating a symptom of TBI in the subject.

26. A kit comprising at least one combination composition according to claim 1 and at least one container.

27. A method of reducing effects from secondary complications of TBI in a subject, comprising: administering to the subject an effective amount of a CtBP expression, translation or activity inhibitor according to claim 1.
